(19) United States
(12) Patent Application Publication
(10) Pub. No.: US 2024/0424016 A1
(43) Pub. Date: Dec. 26, 2024

(54) BUFFERING FOR NON-ALCOHOLIC STEATOHEPATITIS AND LIVER DISEASES

(71) Applicant: DYVE BIOSCIENCES, INC., Camarillo, CA (US)

(72) Inventors: AUDRENE RICE, CAMARILLO, CA (US); NATHAN FITZSIMMONS, CAMARILLO, CA (US)

(21) Appl. No.: 18/826,042

(22) Filed: Sep. 5, 2024

Related U.S. Application Data
(63) Continuation of application No. PCT/US23/17100, filed on Mar. 31, 2023.
(60) Provisional application No. 63/325,617, filed on Mar. 31, 2022.

(51) Int. Cl. A61K 33/00; A61K 9/00; A61K 31/133; A61K 33/10; A61K 45/06; A61K 47/10; A61K 47/12; A61K 47/14; A61K 47/24; A61K 47/26; A61K 47/34; A61K 47/44

(52) U.S. Cl. CPC A61K 33/00; A61K 9/0014; A61K 31/133; A61K 33/10; A61K 45/06; A61K 47/10; A61K 47/12; A61K 47/14; A61K 47/24; A61K 47/26; A61K 47/34; A61K 47/44

(57) ABSTRACT

The present disclosure relates to topical formulations and methods for transdermal delivery of a buffering agent through the skin of a subject. The transdermal delivery treats a liver disease, e.g., a non-alcoholic fatty liver disease (NAFLD) and is a non-alcoholic steatohepatitis (NASH).

### CROSS-REFERENCE TO RELATED APPLICATIONS

This application is a continuation of International Application No. PCT/US23/17100 filed Mar. 31, 2023, which claims the benefit of U.S. Provisional Application No. 63/325,617, filed Mar. 31, 2022, the contents of each of which is incorporated by reference herein in its entirety.

### BACKGROUND

Topical administration describes the application of a substance to a surface of the skin. The term is often used to describe the application of a cream, foam, gel, lotion or ointment to the skin or mucous membranes. The high keratinization of skin cells and their dense packing creates, in most cases, a barrier impermeable to penetration. Because of this, most substances are not absorbed through the skin. There is an unmet need for formulations and method for transdermal penetration that work with a variety of medicaments and active agents, which overcome the barrier presented by the stratum corneum as well as the deeper layers of skin, that do so without harsh solvents, and are effective in delivering high molecular weight agents such as peptides, proteins and nucleic acids.

### SUMMARY

The present disclosure relates to topical formulations and methods for transdermal delivery of a buffering agent through the skin of a subject. The transdermal delivery treats a liver disease, e.g., a non-alcoholic fatty liver disease (NAFLD) and is a non-alcoholic steatohepatitis (NASH).

An aspect of the present disclosure is a method for treating a liver disease or reducing the risk of acquiring a liver disease. The method comprising administering to a subject in need thereof a topical formulation for transdermal delivery of a buffering agent. The topical formulation comprises a penetrant portion and a therapeutically effective amount of the buffering agent. In this aspect, the penetrant portion comprises: a phospholipid, a fatty acid ester formed from a low molecular weight alcohol, and a long-chain fatty acids, and, optionally, one or more of a viscosity-improving agent, a penetration enhancer, an oil, and an emulsifier, and the buffering agent comprises one or more of sodium bicarbonate (baking soda or sodium hydrogen carbonate), tris (tromethamine, trisaminomethane, 2-amino-2-hydroxymethyl-propane-1,3-diol, or tris(hydroxymethyl)aminomethane), calcium carbonate, tripotassium phosphate (potassium phosphate), sodium carbonate (disodium carbonate), sodium hydroxide (sodium oxidanide), potassium bicarbonate (potassium hydrogen carbonate or potassium acid carbonate), lysine, potassium carbonate, dipotassium phosphate (potassium phosphate dibasic or potassium hydrogen phosphate), disodium phosphate (sodium phosphate dibasic or disodium hydrogen phosphate), trisodium phosphate, meglumine ((2r,3r,4r,5s)-6-(methylamino)hexane-1,2,3,4,5-pentol or methylglucamine), arginine, triethanolamine (tea or 2,2′,2″-nitrilotriethanol), glycine, monosodium phosphate (sodium dihydrogen phosphate), monopotassium phosphate (potassium dihydrogen phosphate), monoethanolamine, diethanolamine (diolamine or 2-(2-hydroxyethylamino)ethanol), magnesium carbonate, and 2-imidazole-1-yl-3-ethoxycarbonylpropionic acid (IEPA).

In embodiments, the phospholipid is selected from phosphatidylcholine, hydrogenated phosphatidylcholine, phosphatidylserine, phosphatidylethanolamine, phosphatidylinositol, inositol phosphatide, and sphingomyelin. In some cases, the phospholipid is phosphatidylcholine.

In some embodiments, the penetrant portion comprises two or more phospholipids.

In various embodiments, the phospholipid is in an amount from about 3% to about 15% w/w of the topical formulation.

In additional embodiments, the low molecular weight alcohol is selected from isopropanol, methanol, ethanol, butanol, glycerol, cetyl alcohol.

In embodiments, the low molecular weight alcohol is isopropanol.

In some embodiments, the fatty acid ester is selected from isopropyl palmitate, isopropyl myristate, isopropyl linoleate, isopropyl oleate, ethyl laurate, and ethyl myristate. In some cases, the fatty acid ester is isopropyl palmitate.

In various embodiments, the fatty acid ester is in an amount from about 5% to about 20% w/w of the topical formulation.

In additional embodiments, the fatty acid ester is in an amount from about 7% to about 9% w/w of the topical formulation.

In embodiments, the long-chain fatty acid is selected from a linoleic, oleic, stearic acid, linolenic, palmitic, arachidonic, palmitoleic, myristic, eicosenoic, benehic, euricic, and lignoceric acid. In some cases, the long-chain fatty acid is linoleic acid or oleic acid and/or the long-chain fatty acid is stearic acid.

In some embodiments, the long-chain fatty acid is in an amount from about 0.1% to about 2% w/w of the topical formulation.

In various embodiments, the oil is safflower oil, macadamia oil, almond oil, another oil with high linoleic composition, or another oil with high oleic composition.

In additional embodiments, the oil is in an amount from about 1% to about 7% w/w of the topical formulation.

In embodiments, the oil is almond oil and in an amount from about 2.5% to about 3.5% w/w of the topical formulation.

In some embodiments, the oil is safflower oil and in an amount from about 1.5% to about 2.5% w/w of the topical formulation.

In various embodiments, the penetrant portion comprises a viscosity-improving agent.

In additional embodiments, the viscosity-improving agent is a poloxamer. In some cases, the poloxamer is selected from poloxamer 407, poloxamer 188, poloxamer 184, and poloxamer 124. In some cases, the poloxamer is in an amount from about 4% to about 7% w/w of the topical formulation.

In embodiments, the viscosity-improving agent is polyglyceryl-4 laurate. In some cases, the polyglyceryl-4 laurate is in an amount from about 0.5% to about 2% w/w of the topical formulation. In some cases, the polyglyceryl-4 laurate is in an amount of about 1% w/w of the topical formulation.

In some embodiments, the penetrant portion comprises a penetration enhancer.

In various embodiments, the penetration enhancer is an alcohol or a terpene. In some cases, the penetration enhancer is an alcohol selected from benzyl alcohol, ethanol, propylene glycol, cetyl alcohol, and polyethylene glycol. In some cases, the penetration enhancer comprises two or more alcohols.

In additional embodiments, the penetration enhancer further acts as a preservative.

In embodiments, the penetrant portion comprises two or more penetration enhancers.

In some embodiments, the penetration enhancer is in an amount from about 1% to about 20% w/w of the topical formulation.

In various embodiments, the penetration enhancer is in an amount from about 6% to about 15% w/w of the topical formulation.

In additional embodiments, the penetrant portion comprises at least one penetration enhancer and at least one viscosity-improving agent.

In embodiments, the penetrant portion comprises an emulsifier.

In some embodiments, the emulsifier is selected from polyglyceryl-4-laurate, polyglyceryl-4-oleate, span 60, cetyl alcohol, and polyglyceryl-3-oleate.

In various embodiments, the penetrant portion comprises two or more emulsifiers.

In additional embodiments, the emulsifier is in an amount from about 0.5 to about 10% w/w of the topical formulation.

In embodiments, the penetrant portion comprises at least one emulsifier and at least one viscosity-improving agent.

In some embodiments, the penetrant portion comprises at least one emulsifier and at least one penetration enhancer.

In various embodiments, the penetrant portion comprises at least one emulsifier, at least one viscosity-improving agent, and at least one penetration enhancer.

In additional embodiments, the topical formulation further comprises water. In some cases, the water is in an amount from about 30% water to about 50% w/w of the topical formulation. In some cases, the water is in an amount from about 33% water to about 42% w/w of the topical formulation.

In embodiments, the topical formulation further comprises glucose. In some cases, the glucose is in an amount from about 0.25% to about 0.5% w/w of the topical formulation. In some cases, the glucose is in an amount of about 0.55% w/w of the topical formulation.

In some embodiments, the buffering agent is in an amount from about 25% to about 50% w/w of the topical formulation. In some cases, the buffering agent is in an amount from about 30% to about 35% w/w of the topical formulation. In some cases, the buffering agent is in an amount of about 33% w/w of the topical formulation.

In various embodiments, the formulation is in the form of a cream, lotion, ointment, or the like.

In additional embodiments, the topical formulation increases pH systemically in the subject.

In embodiments, the topical formulation increases the subject's urine pH.

In some embodiments, the liver disease is a non-alcoholic fatty liver disease (NAFLD).

In various embodiments, the liver disease is a non-alcoholic steatohepatitis (NASH).

In additional embodiments, the liver disease is cirrhosis, hepatocellular carcinoma, hepatitis, alcoholic liver disease, hepatic ischemia/reperfusion injury from liver transplant or resection, Budd-Chiari syndrome, primary biliary cholangitis, primary sclerosing cholangitis, progressive familial intrahepatic cholestasis, and Wilson disease.

In embodiments, the subject has cirrhosis or the liver and/or hepatocellular carcinoma (HCC).

In some embodiments, the subject has insulin resistance and/or diabetes, e.g., Type 1 or Type 2 diabetes.

In various embodiments, the subject partakes in a high-fat diet and/or partakes in a high-calorie diet.

In additional embodiments, the subject has a higher than recommended serum cholesterol and/or has a higher than recommended serum triglyceride levels.

In embodiments, the subject has an inflammatory disease and/or chronic inflammation.

In some embodiments, the subject has increased expression of inflammatory cytokines, e.g., Interleukin 1 beta (IL-1β), Tumor Necrosis Factor alpha (TNF-α), Interleukin 6 (IL-6), and Transforming Growth Factor beta (TGF-β).

In various embodiments, the subject has an abnormal gut microbiota.

In additional embodiments, the subject is additionally administered a therapeutic for treating the liver disease or a related disorder. In some cases, the therapeutic for treating the liver disease or a related disorder is administered before, contemporary with, or after the topical formulation. In some cases, the therapeutic is administered orally, topically, enterally, parenterally, by intravenous injection or infusion, by intraperitoneal injection, by intramuscular injection, or by subcutaneous injection. In some cases, the therapeutic is a liquid, a suspension, a gel, a geltab, a semisolid, a tablet, a sachet, a lozenge, a pill, or a capsule. In some cases, the therapeutic is a drug relating to glycemic control (e.g., a sodium-glucose cotransporter-2 (SGLT2) inhibitor), a drug for lipid metabolism (e.g., a fibrate), and/or a NASH drug that has failed in a clinical trial due to lack of efficacy (e.g., elafibranor, cenicriviroc, remoglifozin, Obeticholic, Resmetirom, and Nalmefene).

In embodiments, the subject is additionally administered a therapeutic for treating the liver disease or a related disorder, with the therapeutic being included in the topical formulation. In some cases, transdermal delivery of the therapeutic via the topical formulation provides systemic administration of the therapeutic. In some cases, the therapeutic is a drug relating to glycemic control (e.g., a sodium-glucose cotransporter-2 (SGLT2) inhibitor), a drug for lipid metabolism (e.g., a fibrate), and/or a NASH drug that has failed in a clinical trial due to lack of efficacy (e.g., elafibranor, cenicriviroc, remoglifozin, Obeticholic, Resmetirom, and Nalmefene).

In some embodiments, the topical formulation comprises about 33% sodium bicarbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate.

In various embodiments, the topical formulation comprises about 33% calcium carbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate.

In additional embodiments, the topical formulation comprises about 33% sodium bicarbonate, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate.

In embodiments, the topical formulation comprises about 33% tris, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate.

In various embodiments, the subject in need thereof is selected for having a liver disease or at risk for having a liver disease. In some cases, the subject in need thereof is not administered a topical formulation of the present disclosure for transdermal delivery of a buffering agent before being selected for having a liver disease or at risk for having a liver disease. In other words, the subject had not previously been administered a topical formulation of the present disclosure. Thus, the subject in need is specifically selected to be administered ministered a topical formulation of the present disclosure due a diagnosis of having a liver disease or at risk for having a liver disease.

Another aspect of the present disclosure is a topical formulation for transdermal delivery of a buffering agent through the skin of a subject. The topical formulation comprising one or more buffering agents, one or more fatty acid esters, one or more alcohols, one or more long-chain fatty acids, one or more oils, one or more poloxamers, one or more phospholipids, polyglyceryl-4 laurate, and water.

In some embodiments, the one or more alcohols are present in an amount from about 1% to about 13% w/w of the topical formulation. In some cases, the one or more alcohols are selected from benzyl alcohol, ethanol, cetyl alcohol, and propylene glycol. When present benzyl alcohol may be in an amount from about 1% to about 2% w/w of the topical formulation; when present ethanol may be in an amount from about 1% to about 2% w/w of the topical formulation; when present cetyl alcohol may be in an amount from about 1.5% to about 2.5% w/w of the topical formulation; and when present propylene glycol may be in an amount from about 4% to about 6% w/w of the topical formulation.

In various embodiments, the one or more fatty acid esters are present in an amount from about 6% to about 10% w/w of the topical formulation.

In additional embodiments, the one or more fatty acid esters are present in an amount from about 7% to about 9% w/w of the topical formulation.

In embodiments, the one or more fatty acid esters is selected from isopropyl palmitate and isopropyl myristate.

In some embodiments, the one or more long-chain fatty acids are present in an amount from about 0.3% to about 2% w/w of the topical formulation.

In various embodiments, the one or more long the long-chain fatty acids are selected from a linoleic, oleic, stearic acid, linolenic, palmitic, arachidonic, palmitoleic, myristic, eicosenoic, benehic, euricic, and lignoceric acid. When present stearic acid may be in an amount from about 0.3% to about 0.5% w/w of the topical formulation and when present oleic acid may be in an amount from about 0.4% to about 0.6% w/w of the topical formulation.

In additional embodiments, the one or more oils are present in an amount from about 1% to about 7% w/w of the topical formulation.

In embodiments, the one or more oils are safflower oil, macadamia oil, almond oil, another oil with high linoleic composition, or another oil with high oleic composition. When present safflower oil may be in an amount from about 1.5% to about 2% w/w of the topical formulation and when present almond oil may be in an amount from about 2.5% to about 3.5% w/w of the topical formulation.

In some embodiments, the one or more poloxamers are in an amount from about 4% to about 7% w/w of the topical formulation.

In various embodiments, the one or more poloxamers are in an amount from about 5% to about 6.5% w/w of the topical formulation.

In additional embodiments, the one or more poloxamers are selected from poloxamer 407, poloxamer 188, poloxamer 184, and poloxamer 124.

In embodiments, the one or more phospholipids are in an amount from about 3.5% to about 4.5% w/w of the topical formulation.

In some embodiments, the one or more phospholipids are selected from phosphatidylcholine, hydrogenated phosphatidylcholine, phosphatidylserine, phosphatidylethanolamine, phosphatidylinositol, inositol phosphatide, and sphingomyelin.

In various embodiments, the polyglyceryl-4 laurate is in an amount from about 0.5% to about 1.5% w/w of the topical formulation.

In additional embodiments, the polyglyceryl-4 laurate is in an amount of about 1% w/w of the topical formulation.

In embodiments, the water is in an amount from about 30% to about 42% w/w of the topical formulation.

In some embodiments, the one or more buffering agents are in an amount from about 25% to about 40% w/w of the topical formulation.

In various embodiments, the one or more buffering agents are in an amount from about 30% to about 35% w/w of the topical formulation.

In additional embodiments, the one or more buffering agents are in an amount of about 33% w/w of the topical formulation.

In embodiments, the one or more buffering agents are selected from sodium bicarbonate (baking soda or sodium hydrogen carbonate), tris (tromethamine, trisaminomethane, 2-amino-2-hydroxymethyl-propane-1,3-diol, or tris(hydroxymethyl)aminomethane), calcium carbonate, tripotassium phosphate (potassium phosphate), sodium carbonate (disodium carbonate), sodium hydroxide (sodium oxidanide), potassium bicarbonate (potassium hydrogen carbonate or potassium acid carbonate), lysine, potassium carbonate, dipotassium phosphate (potassium phosphate dibasic or potassium hydrogen phosphate), disodium phosphate (sodium phosphate dibasic or disodium hydrogen phosphate), trisodium phosphate, meglumine ((2r,3r,4r,5s)-6-(methylamino)hexane-1,2,3,4,5-pentol or methylglucamine), arginine, triethanolamine (tea or 2,2′,2″-nitrilotriethanol), glycine, monosodium phosphate (sodium dihydrogen phosphate), monopotassium phosphate (potassium dihydrogen phosphate), monoethanolamine, diethanolamine (diolamine or 2-(2-hydroxyethylamino)ethanol), magnesium carbonate, and 2-imidazole-1-yl-3-ethoxycarbonylpropionic acid (IEPA).

In some embodiments, the topical formulation comprises about 33% sodium bicarbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate.

In various embodiments, the topical formulation comprises about 33% calcium carbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate.

In additional embodiments, the topical formulation comprises about 33% sodium bicarbonate, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate.

In embodiments, the topical formulation comprises about 33% tris, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate.

Yet another aspect of the present disclosure is a method for treating a liver disease or reducing the risk of acquiring a liver disease, the method comprising a step of applying to the skin of a subject an effective amount of any herein-disclosed topical formulation.

In some embodiments, the liver disease is a non-alcoholic fatty liver disease (NAFLD) with or without non-alcoholic steatohepatitis (NASH).

In various embodiments, the liver disease is cirrhosis, hepatocellular carcinoma, hepatitis, alcoholic liver disease, hepatic ischemia/reperfusion injury from liver transplant or resection, Budd-Chiari syndrome, primary biliary cholangitis, primary sclerosing cholangitis, progressive familial intrahepatic cholestasis, Wilson disease, and/or hepatocellular carcinoma (HCC).

In additional embodiments, the subject is additionally administered a therapeutic for treating the liver disease or a related disorder. In some cases, the therapeutic for treating the liver disease or a related disorder is administered before, contemporary with, or after the topical formulation. In some cases, the therapeutic is administered orally, topically, enterally, parenterally, by intravenous injection or infusion, by intraperitoneal injection, by intramuscular injection, or by subcutaneous injection. In some cases, the therapeutic is a liquid, a suspension, a gel, a geltab, a semisolid, a tablet, a sachet, a lozenge, a pill, or a capsule. In some cases, the therapeutic is a drug relating to glycemic control (e.g., a sodium-glucose cotransporter-2 (SGLT2) inhibitor), a drug for lipid metabolism (e.g., a fibrate), and/or a NASH drug that has failed in a clinical trial due to lack of efficacy (e.g., elafibranor, cenicriviroc, remoglifozin, Obeticholic, Resmetirom, and Nalmefene).

In some embodiments, the subject is additionally administered a therapeutic for treating the liver disease or a related disorder, with the therapeutic being included in the topical formulation. In some cases, transdermal delivery of the therapeutic via the topical formulation provides systemic administration of the therapeutic. In some cases, the therapeutic is a drug relating to glycemic control (e.g., a sodium-glucose cotransporter-2 (SGLT2) inhibitor), a drug for lipid metabolism (e.g., a fibrate), and/or a NASH drug that has failed in a clinical trial due to lack of efficacy (e.g., elafibranor, cenicriviroc, remoglifozin, Obeticholic, Resmetirom, and Nalmefene).

In various embodiments, the subject in need thereof is selected for having a liver disease or at risk for having a liver disease. In some cases, the subject in need thereof is not administered a topical formulation of the present disclosure for transdermal delivery of a buffering agent before being selected for having a liver disease or at risk for having a liver disease. In other words, the subject had not previously been administered a topical formulation of the present disclosure. Thus, the subject in need is specifically selected to be administered ministered a topical formulation of the present disclosure due a diagnosis of having a liver disease or at risk for having a liver disease.

A further aspect of the present disclosure is the use of any herein-disclosed topical formulation in a method for treating a liver disease, reducing the risk of acquiring a liver disease, or reducing a symptom of a liver disease.

A topical formulation for transdermal delivery of a buffering agent comprises mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize one or more distinct permeation enhancers.

Any aspect or embodiment described herein can be combined with any other aspect or embodiment as disclosed herein.

### DETAILED DESCRIPTION

The present disclosure relates to topical formulations and methods for transdermal delivery of a buffering agent through the skin of a subject. The transdermal delivery treats a liver disease, e.g., a non-alcoholic fatty liver disease (NAFLD) and is a non-alcoholic steatohepatitis (NASH).

### Introduction

The term transdermal administration refers to applying a substance onto the skin so that it is absorbed into the body for local or systemic distribution. A transdermal solution (e.g., cream, ointment, or lotion) or transdermal patch is typically placed on one's skin. The solution or patch includes a medicament that is released into the skin. As the layers of skin absorb the solution, the medicament is absorbed via the blood vessels into the bloodstream. From there, the substance can be circulated through the body.

There are clear advantages to transdermal administration of medicaments. The consumer does not have to schedule and remember to consume doses of pills. Further, transdermal administration is not affected by stomach or digestive issues. Administration across the skin enables drugs to avoid degradation in the gastrointestinal tract or liver. Transdermal delivery is therefore of particular interest for molecules with limited systemic bioavailabilities and short half-lives. Drugs that are absorbed slowly can be more effective. With a transdermal patch or cream, a medicament can be released in small quantities over a long period of time.

Transdermal administration can be effective in administering hydrophobic chemicals such as steroid hormones. For example, transdermal patches are a common means of administering steroidal drugs for birth control, hormone replacement therapy and prevention of motion sickness. Common medicaments that can be administered by transdermal patches include pain relievers, nicotine, hormones, and drugs to treat angina and motion sickness.

Medicaments that are not hydrophobic chemicals are typically unsuited for topical administration. To be effective, the active drug or agent in a topical composition must penetrate the skin, which is structurally complex and relatively thick. Molecules moving through the skin must first penetrate the stratum corneum and any material on its surface. The molecules must then penetrate the epidermis, the papillary dermis, and the capillary walls into the vascular system or lymphatic system. To be absorbed, the molecules must overcome a different resistance to penetration in each layer.

Strategies have been devised to improve transdermal administration of medicaments. These strategies can be categorized as either physical, chemical, mechanical, or biochemical. Combinations of these strategies can also increase efficacy or extend the time for transdermal delivery. Physical techniques include abrasion and tape stripping, which physically break open the skin. Another physical method is prolonged occlusion, which alters the barrier properties of the stratum corneum. After 24 to 28 hours of occlusion with resultant hydration, corneocytes swell, intercellular spaces become distended, and the lacunar network becomes dilated. Distention of the lacunae eventually leads to connections with an otherwise discontinuous system. This creates pores in the stratum corneum interstices through which polar and non-polar substances can penetrate more easily.

Other approaches include the use of Chemical Permeation Enhancers. Chemical Permeation Enhancers (CPEs) are molecules that interact with the constituents of skin's outermost layer, the stratum corneum (SC), and increase its permeability. However, despite efforts at improving them, CPEs are minimally effective in increasing the rate at which drugs permeate the skin. CPEs can also cause skin damage, irritation and sensitization. Further, they are generally ineffective with high molecular weight drugs such as peptides, proteins and nucleic acids.

Although a variety of methods can be used to enhance transdermal drug delivery, these methods have limitations. Most efforts to enhance transdermal penetration have focused on the outermost layer of the skin, the stratum corneum. They typically rely on harsh solvents (e.g., alcohols, DMSO) or patch-based systems. This approach limits the molecular size, lipophilicity, and potency of drugs that can be used. In essence, current approaches are largely limited to small, lipophilic, and highly potent drugs.

### Non-Alcoholic Fatty Liver Diseases

Non-alcoholic fatty liver disease (NAFLD) is a type of chronic liver disease characterized by excess lipids in the liver. Roughly 15 to 20% of NAFLD cases progress to non-alcoholic steatohepatitis (NASH). NASH is an increasingly prevalent liver disease characterized by progressive steatosis, inflammation, and fibrosis. These pathological processes may contribute to acidification of the local environment and local acidification may exacerbate the disease state and promote disease progression, creating a vicious cycle. Neutralization of the acidic microenvironment with a buffering agent may help to slow, stall, or even reverse disease progression, improving patient prognosis. The compositions and methods of the present disclosure interrupt this vicious cycle by addressing local acidification in NAFLD, e.g., NASH, to reduce and/or ameliorate disease pathophysiology and provide a therapeutic effect. Unfortunately, there does not exist an approved treatment for NASH.

In NASH, steatosis, which is the accumulation of lipids in the liver, is accompanied by fibrosis and an inflammatory response. If left untreated, NASH can progress to cirrhosis which may ultimately lead to liver failure or the development of hepatocellular carcinoma—the second leading cause of cancer-related death worldwide in 2018. However, an FDA approved treatment for NASH does not yet exist, and the occurrences are rising due to the strong association with increasingly prevalent metabolic disorders such as obesity and type II diabetes. In 2016, over 64 million patients were estimated to have NAFLD in the US, with an associated clinical cost of approximately $103B. Models predict a total of 100.9 million cases of NAFLD in the US in 2030, accompanied by approximately 27 million cases of NASH.

There is some evidence to suggest a connection between NAFLD, e.g., NASH, and systemic acidosis. Several metabolic diseases, including dyslipidemia and diabetes, are associated with systemic acidosis and decreased urine pH.

The liver plays an important role in acid-base regulation and can compensate for imbalances to produce a net neutral result. For example, patients with acute liver failure suffer from extreme lactic acidosis, yet this is counteracted by hypooalbuminemic alkalosis resulting in a normal pH. The overall pH appears normal; however, lactate, albumin, and bicarbonate ions are outside of their normal ranges resulting in reduced buffering capacity and increased susceptibility to acute pH fluctuations. In NASH in particular, there are aspects of the pathophysiology that may contribute to the development of an acidic microenvironment.

The characteristic pathophysiological processes in NASH may each contribute to the development of an acidic microenvironment. Steatosis, caused by an accumulation of lipids in the liver, is linked to insulin resistance in the liver and adipose tissue. Insulin resistance results in a metabolic shift towards ketosis for energy, which results in increased production of acidic ketone bodies.

The inflammatory process may contribute to extracellular acidification. The infiltration and inflammatory activity of immune cells results in a switch towards glycolytic metabolism resulting in increased lactic acid production. Inflammatory stimuli can promote fibrosis in hepatic stellate cells and activate ion exchange proteins in the cell membrane which pump acidifying hydrogen ions into the extracellular environment. Finally, tissue injury caused by lipotoxicity and fibrosis progression can also contribute to proton accumulation resulting in further reduced pH. NASH pathophysiology may promote an acidic microenvironment in multiple ways and, without wishing to be bound by theory, acidity may encourage NASH disease progression, thereby, creating a vicious cycle. The compositions and methods of the present disclosure interrupt this vicious cycle by addressing local acidification in NASH to reduce and/or ameliorate disease pathophysiology and provide a therapeutic effect.

A high-fat diet is associated with the accumulation of free fatty acids and cholesterol in the liver. Approximately 60% of the liver mass is composed of hepatocytes—the primary functional cells in the liver. Accumulating free cholesterol has been identified in hepatocytes of patients with NASH, and excess free cholesterol is linked to fibrosis progression and innate immune system activation. An unhealthy diet can incite metabolic defects to the extent that a high-fat, high-calorie feeding regimen is often used to induce NASH-related pathologies in animal models. It is becoming increasingly recognized that gut microbiota is involved in metabolic regulation and therefore play a role in the pathogenesis of NAFLD. Patients with NAFLD have been shown to have a small degree of intestinal bacterial overgrowth as well as increased gut permeability. Overgrowth of gut bacteria is associated with increased lipopolysaccharide (LPS), an endotoxin derived from the bacterial cell wall. When bacterial overgrowth is couple with increased gut permeability, translocation of LPS from the intestines into systemic circulation occurs, ultimately inducing stress and igniting an inflammatory response in the liver.

De novo lipogenesis is a process in which glucose and fructose are enzymatically converted to lipids in hepatocytes and adipose tissue. This is a natural, regulatory metabolic process that allows for excess carbohydrates to ultimately be converted to triglycerides for storage. However, if the rate of lipogenesis exceeds the rate of lipolysis, or lipid breakdown, in the liver then steatosis begins to develop.

In NASH, the excessive accumulation of lipids disrupts homeostasis resulting in stress conditions. Endoplasmic reticulum (ER) stress, in particular, is an identifying characteristic of NASH. The ER is an organelle within the cell that serves as a quality control checkpoint for synthesized proteins before they are exported. ER stress in hepatocytes upregulates lipogenesis and limits triglyceride secretion, further contributing to the development of lipotoxicity (fat-induced liver toxicity). Delayed or insufficient responses to ER stress can result in exacerbated pathologies ultimately leading to inflammation and apoptosis (cell death). ER stress is therefore hypothesized to play a crucial role in the progression of NAFLD to NASH.

Insulin resistance in the liver and adipose tissue is a metabolic dysfunction that is crucially linked to NAFLD progression. Studies in mice have shown that a high-fat diet can induce insulin resistance in tissues within 3 to 7 days. In adipose tissue, normal insulin signaling inhibits triglyceride lipolysis allowing for triglycerides to be maintained as stored energy while glucose is available. However, insulin resistance disrupts this signaling pathway leading to increased lipolysis (breakdown) of triglycerides in the adipocytes and the release of free fatty acids into circulation.

Circulating free fatty acids are then processed in the liver in one of three ways: mitochondrial beta-oxidation to produce energy, incorporation into other functional lipids such as phospholipids or ceramides, or esterification to form triglycerides for storage. If the fatty acids are in excess of what the liver can process for energy, they are stored as lipid droplets in the hepatocytes or exported as triglycerides via very low-density lipoproteins. Dysregulation of lipid droplet formation and breakdown can result in lipotoxicity and hepatocellular ballooning, another unique identifier of NASH. Hepatocellular ballooning is the enlargement of cells due to the accumulation of small fat droplets, resulting in displacement of the nucleus to the cell periphery, dilation of the endoplasmic reticulum, and low cytosolic pressure.

Steatosis alone is a characteristic of NAFLD which does not pose the same risk to patient health; however, mechanisms which provoke steatosis, such as ER stress, can also incite inflammatory and fibrotic processes. When the primary insult of steatosis is accompanied by other pathogenic drivers, the disease state progresses towards NASH. One mechanism by which this occurs is when lipotoxicity and ER stress induce hepatocellular ballooning leading to hepatocyte damage and cell death. When cells undergo apoptosis (cell death), the cellular contents are released and act as damage associated molecular patterns (DAMPs) which activate pattern recognition receptors (PRRs) and trigger an inflammatory response.

Similar to NASH, the type 2 diabetes pathophysiology is characterized by chronic inflammation, hyperlipidemia, extracellular matrix disorders and altered microbiota. An acidic local and systemic pH may cause insulin resistance, e.g., in subjects with type 2 diabetes or pre-diabetes, by reducing the affinity of insulin for its cellular receptor.

Administration of buffering agent may reduce insulin resistance in the liver and adipose tissue, halting triglyceride lipolysis and the resulting influx of free fatty acids in the liver. With triglyceride lipolysis contributing to approximately 60% of excess lipids in NASH, slowing or inhibiting triglyceride lipolysis by reestablishing the insulin signaling pathway could have a meaningful impact on a primary driver of steatosis. Furthermore, removing insulin resistance and the lipotoxic pressure it brings may also decrease the hepatocyte stress stimuli that promote inflammation and fibrosis in NASH.

Lipotoxicity and resulting hepatocyte stress and injury can ignite a multifaceted immune response. Lipotoxicity-induced ER stress activates the NF-1B and JNK inflammatory pathways resulting in inflammatory cytokine production. Additionally, hepatocyte damage and death caused by lipotoxicity can initiate the innate immune response further promoting inflammation and disease progression. Innate immune mechanisms not only contribute to inflammation but also are suggested to affect steatosis, insulin resistance, and fibrosis.

Pro-inflammatory cytokines are secreted by immune cells and signal the recruitment, activation, and inflammatory response of other immune cells in the tissue. Inflammatory cytokines perform an important role of initiating and coordinating the body's inflammatory response to injury or infection. However, when produced chronically and excessively, inflammatory cytokines can be more damaging than the inciting event itself.

The most prominent inflammatory cytokines in NASH pathology are Interleukin 1 beta (IL-1β), Tumor Necrosis Factor alpha (TNF-α), Interleukin 6 (IL-6), and Transforming Growth Factor beta (TGF-β). IL-1β is the most potent in promoting inflammation and disease progression. It acts as a key mediator of the immune and inflammatory responses by promoting the recruitment and adherence of inflammatory cells at the site of inflammation. Furthermore, secretion of IL-1β promotes lipid droplet formation and cholesterol accumulation in hepatocytes, induces IL-6 production, and stimulates HSC activation, contributing to each of the characterizing NASH pathologies.

TNF-α has the second largest impact on NASH disease progression. It is a cytokine and adipokine, meaning it is produced by both immune cells and adipocytes. Kupffer cells are the most common source of TNF-α, and upon release it signals other immune cells to initiate and regulate an inflammatory response. This regulatory signaling can involve inducing apoptotic cell death of damaged or diseased cells. When released by adipocytes, TNF-α influences adipose tissue metabolism and promotes insulin resistance. TNF-α can induce a pro-inflammatory phenotype in macrophages, apoptosis in hepatocytes, and a fibrogenic phenotype in hepatic stellate cells (HSCs). Consequently, excessive TNF-α production has been implicated in a broad range of disease states.

Interleukin-6, primarily released by Kupffer cells, induces the acute phase response in hepatocytes. The acute phase response occurs soon after the initiation of the inflammatory process to protect against tissue injury and promote tissue repair. In hepatocytes, it involves the upregulation of acute phase protein synthesis including C-reactive protein, which is a widely accepted biomarker for inflammation.

Lastly, TGF-β regulates cell survival, proliferation, and fibrosis. In particular, it activates HSCs to adopt the fibrogenic phenotype and contributes to lipid accumulation and apoptosis in hepatocytes, all of which promote steatosis and inflammation resulting in NASH disease progression.

The primary functions of hepatocytes are metabolism, detoxification, and protein synthesis. When stressed, hepatocytes can also assume an immune function, sensing DAMPs and PAMPs and triggering local and systemic inflammatory responses. ER stress caused by disrupted hepatocyte homeostasis, as well as increased levels of LPS, is linked to the release of pro-inflammatory extracellular vesicles. The contents of these vesicles communicate the need for an inflammatory response to neighboring cells. Additionally, hepatocytes can synthesize proteins which can be transported beyond the anatomical borders of the liver, extending the inflammatory response to other organs and tissues in the body.

Stressed hepatocytes can induce activation of inflammatory pathways that are critical in NASH disease progression: NF-κB and JNK. Nuclear factor-kappa B (NF-κB) is a transcription factor involved in innate and adaptive immune responses, inflammation, and hepatic insulin sensitivity. Activation of the NF-κB pathway results in the release of pro-inflammatory cytokines including TNF-α and IL-6, which promote macrophage polarization to the pro-inflammatory phenotype.

Interleukin-6 (IL-6) also activates the JNK pathway, which inhibits insulin signaling and suppresses insulin secretion from the pancreas. Therefore, both NF-κB and JNK activation contribute to increased insulin resistance. This promotes triglyceride accumulation and increased lipotoxicity which induces additional hepatocyte stress and damage, igniting a perpetual inflammatory cycle.

Lipotoxicity induces hepatocyte damage and ultimately cell death. When hepatocytes undergo apoptosis, their cellular contents act as DAMPs which serve as a danger signal to other cells in the liver including mast cells and Kupffer cells, initiating the innate immune response. The presence of DAMPs triggers mast cells to release pro-inflammatory cytokines including TNF-α, TGF-β, and IL-6 which play an important role in macrophage polarization.

Kupffer cells are the resident macrophage in the liver, and they account for approximately 10-15% of liver cells. Macrophages are a type of white blood cell that are responsible for eliminating foreign substances and initiating immune and inflammatory responses. Macrophages exist between two polarization states-M1 and M2. M1 polarization is associated with a pro-inflammatory phenotype whereas M2 polarization is anti-inflammatory.

In response to the apoptosis-related danger signals, Kupffer cells are activated resulting in a shift towards the M1 pro-inflammatory phenotype. This phenotypic change in the Kupffer cell population promotes a variety of other responses in the tissue, increasing the liver predisposition to steatosis as well as HSC activation and extracellular matrix remodeling lending to fibrosis. Additionally, activated Kupffer cells aggregate and signal the infiltration of bone marrow-derived monocytes and other leukocytes into the liver. As a result, the immune cell composition of the liver shifts significantly.

Together the Kupffer cells, bone marrow-derived monocytes, mast cells, and other leukocytes contribute to the persistent inflammation observed in NASH. The release of pro-inflammatory cytokines coupled with continuing hepatocyte damage and DAMP signaling results in increased IL-1β secretion from Kupffer cells and bone marrow-derived monocytes. Interleukin-1β is the most potent pro-inflammatory cytokine in NASH and is known to promote the progression of liver steatosis, inflammation, and fibrosis through a variety of mechanisms.

Macrophage polarization to the inflammatory phenotype is promoted by LPS in the liver and inflammatory cytokines such as TNF-α. Kupffer cell activation is associated with increased secretion of IL-1β. IL-1β expression is upregulated in Kupffer cells via inflammasome activation. The NLRP3 inflammasome is an intracellular multiprotein complex linked to the pattern recognition receptors that respond to DAMPs. NLRP3 expression is low in healthy hepatocytes and is increased in animal models and humans with NASH. When activated in Kupffer cells, the NLRP3 inflammasome upregulates the production of IL-1β and other pro-inflammatory cytokines.

Transient receptor potential vanilloid-type 4 (TRPV4) is a calcium ion channel expressed in tissues and immune cells and is implicated in the activation of innate immune cells such as monocytes and macrophages. TRPV4 is activated by a variety of stimuli including DAMPs and collagen from injured hepatocytes. TRPV4 activation has been linked to increased IL-1β expression from bone marrow derived monocytes. Some of the inflammatory response observed in NASH is regulated through TRPV4 activation, however it is also hypothesized that TRPV4 plays a crucial role in fibrosis.

The infiltration and activation of inflammatory cells leads to increased energy and oxygen demand, accelerated glycolysis, and increased lactic acid production resulting in local acidification. Acidosis may be stressor for cells and has been implicated in apoptosis, inflammation, and immune cell response. A low extracellular pH may lead to both a macrophage M1 polarization and a persistent inflammatory response, which may operate by activating the NLRP3 inflammasome. As previously mentioned, NLRP3 activation induces macrophage production and secretion of pro-inflammatory cytokines, namely IL-1β.

Acidity may also be linked to activation of the ER stress response pathway resulting in stimulation of the NF-κB and JNK inflammatory pathways in macrophages and monocytes. The net effect of the ER stress response and NLRP3 inflammasome activations is increased expression of IL-6, IL-1β, and TNF-α, which together activate local immune cells leading to a chronic inflammatory state.

Inflammation is a normal response to cell damage and disease. However, uncontrolled and chronic inflammation coupled with other pathogenic drivers in NASH can lead to fibrosis. As previously mentioned, the onset of inflammation is characterized by the release of several pro-inflammatory mediators including TNF-α, TGF-β, and IL-1β. These pro-inflammatory cytokines also play a direct role in promoting fibrosis.

Fibrosis is the excessive accumulation of extracellular matrix proteins. Hepatic stellate cells (HSCs) are the primary collagen producing cells in the liver. When activated, HSCs undergo a phenotypic shift towards a proliferative, pro-inflammatory, and fibrogenic cell type. The activated HSCs secrete extracellular matrix (ECM) components including α-smooth muscle actin and collagen. The accumulation of these ECM proteins distorts the structure of the liver and forms a fibrous scar. If not controlled, fibrosis can progress to cirrhosis resulting in hepatocellular dysfunction, resistance to blood flow, and ultimately liver failure.

Progressive NASH is characterized by stressed or apoptotic hepatocytes and M1-polarized macrophages signaling the activation of HSCs. Injured hepatocytes and Kupffer cells activate HSCs via TRPV4. While the exact mechanism of TRPV4 activation is yet to be understood, it is known that TRPV4 is responsive to DAMPs released by injured hepatocytes. In a murine model of induced liver fibrosis, activation of TRPV4 channels was associated with an increased severity in liver fibrosis while pharmacological inhibition and genetic deletion of TRPV4 resulted in the alleviation of liver fibrosis in vivo. This suggests that TRPV4 plays a crucial role in fibrosis and may be a viable therapeutic target.

HSC activation has also been linked to osteopontin, which a multifunctional protein secreted in the liver. Osteopontin (OPN) is considered a double-edged sword in fatty liver disease, as it is believed to hold both protective and deleterious functions. Elevated expression of OPN in NASH patients, however, has correlated with triglyceride accumulation and fibrosis progression. While the mechanism of OPN influence on HSCs is not fully elucidated, neutralization of OPN with interfering RNA in mice resulted in an attenuated fibrogenic response, highlighting the key role osteopontin must play in fibrosis.

Several factors are believed to promote fibrosis including inflammation and ECM stiffening due to matrix remodeling. HSCs are activated by TNF-α and TGF-β, which are released by mast cells and Kupffer cells when the innate immune response is triggered and by stressed hepatocytes when the JNK inflammatory pathway is stimulated. HSC activation encourages prolific ECM production and remodeling resulting in matrix stiffening. HSCs are constantly sensing and responding to matrix stiffness, and an increase in matrix stiffness has been shown to upregulate ECM production. Persistent ECM production continues to stiffen the extracellular matrix, creating a self-promoting cycle of fibrosis progression.

ECM stiffening has also been linked to macrophage M1 polarization resulting in a heightened inflammatory response. This was supported by a study which showed a correlation between cases of advanced fibrosis and increasing levels of TNF-α, a key cytokine in promoting Kupffer cell polarization. Interestingly, Dutta et al. showed that TRPV4, which mediates bone marrow-derived monocyte and HSC activation, also mediates macrophage M1 polarization stimulated by increased ECM stiffness.

Finally, in addition to promoting macrophage polarization through ECM production, activated HSCs release pro-inflammatory cytokines which stimulate an inflammatory response from resident leukocytes. This creates a positive feedback loop in which activated fibrogenic and inflammatory cells are continuously stimulating one another to release pro-inflammatory cytokines, contributing to the chronic inflammatory response observed in NASH.

A key mediator of HSC activation and fibrosis progression is TRPV4. TRVP4 can be activated by a variety of stimuli including DAMPs, hypertonicity, and low pH. An acidic microenvironment may contribute to TRPV4 activation, resulting in amplified HSC transition to the fibrogenic phenotype as well as activation of bone marrow-derived monocyte cells and Kupffer cells resulting in IL-1β secretion. Once fibrosis starts, there are multiple feedback loops that continually promote chronic inflammation, insulin resistance, and lipotoxicity. Therefore, normalization of the local pH with a buffering agent may reduce or inhibit TRPV4 activation leading to decreased HSC activation and resulting fibrosis, ECM stiffening, and inflammatory involvement.

Non-alcoholic steatohepatitis is an increasingly prevalent disease with a complex pathophysiology involving steatosis, inflammation, and fibrosis. The spaciotemporal dynamics of these three components of NASH pathology are not fully understood. However, it seems as though targeting a single pathway is not sufficient to alter disease progression. As of 2020, at least 10 compounds have been developed for NASH and proceeded through clinical trials yet have failed to make it to market. The difficulty in identifying a successful treatment may be due to the multifaceted and intertwined pathophysiology.

Without wishing to be bound by theory, it appears that an acidic microenvironment may be associated with NASH pathophysiology and that this local acidity may contribute to disease progression. Acidity appears to play a role in all three manifestations of NASH pathophysiology: steatosis, inflammation, and fibrosis. Therapeutic mechanisms targeting this acidic microenvironment, such as neutralization with a buffering agent, may allow for a multi-targeted approach to treatment. Accordingly, the present disclosure relates to formulations and methods for treating a liver disease, e.g., NASH therapeutic.

Notably, a subject in need thereof is selected for having a liver disease or at risk for having a liver disease. In some cases, the subject in need thereof is not administered a topical formulation of the present disclosure for transdermal delivery of a buffering agent before being selected for having a liver disease or at risk for having a liver disease. In other words, the subject had not previously been administered a topical formulation of the present disclosure. Thus, the subject in need is specifically selected to be administered ministered a topical formulation of the present disclosure due a diagnosis of having a liver disease or at risk for having a liver disease.

### Illustrative Aspects of the Present Disclosure

An aspect of the present disclosure is a method for treating a liver disease or reducing the risk of acquiring a liver disease. The method comprising administering to a subject in need thereof a topical formulation for transdermal delivery of a buffering agent. The topical formulation comprises a penetrant portion and a therapeutically effective amount of the buffering agent. In this aspect, the penetrant portion comprises: a phospholipid, a fatty acid ester formed from a low molecular weight alcohol, and a long-chain fatty acids, and, optionally, one or more of a viscosity-improving agents, a penetration enhancer, an oil, and an emulsifier, and the buffering agent comprises one or more of sodium bicarbonate (baking soda or sodium hydrogen carbonate), tris (tromethamine, trisaminomethane, 2-amino-2-hydroxymethyl-propane-1,3-diol, or tris(hydroxymethyl)aminomethane), calcium carbonate, tripotassium phosphate (potassium phosphate), sodium carbonate (disodium carbonate), sodium hydroxide (sodium oxidanide), potassium bicarbonate (potassium hydrogen carbonate or potassium acid carbonate), lysine, potassium carbonate, dipotassium phosphate (potassium phosphate dibasic or potassium hydrogen phosphate), disodium phosphate (sodium phosphate dibasic or disodium hydrogen phosphate), trisodium phosphate, meglumine ((2r,3r,4r,5s)-6-(methylamino)hexane-1,2,3,4,5-pentol or methylglucamine), arginine, triethanolamine (tea or 2,2′,2″-nitrilotriethanol), glycine, monosodium phosphate (sodium dihydrogen phosphate), monopotassium phosphate (potassium dihydrogen phosphate), monoethanolamine, diethanolamine (diolamine or 2-(2-hydroxyethylamino)ethanol), magnesium carbonate, and 2-imidazole-1-yl-3-ethoxycarbonylpropionic acid (IEPA).

Another aspect of the present disclosure is a topical formulation for transdermal delivery of a buffering agent through the skin of a subject. The topical formulation comprising one or more buffering agents, one or more fatty acid esters, one or more alcohols, one or more long-chain fatty acids, one or more oils, one or more poloxamers, one or more phospholipids, polyglyceryl-4 laurate, and water.

Yet another aspect of the present disclosure is a method for treating a liver disease or reducing the risk of acquiring a liver disease, the method comprising a step of applying to the skin of a subject an effective amount of any herein-disclosed topical formulation.

A further aspect of the present disclosure is the use of any herein-disclosed topical formulation in a method for treating a liver disease, reducing the risk of acquiring a liver disease, or reducing a symptom of a liver disease.

In some cases, a subject in need thereof is selected for having a liver disease or at risk for having a liver disease. The subject in need thereof is not administered a topical formulation of the present disclosure for transdermal delivery of a buffering agent before being selected for having a liver disease or at risk for having a liver disease. In other words, the subject had not previously been administered a topical formulation of the present disclosure. Thus, the subject in need is specifically selected to be administered ministered a topical formulation of the present disclosure due a diagnosis of having a liver disease or at risk for having a liver disease.

Any aspect or embodiment described herein can be combined with any other aspect or embodiment as disclosed herein.

Certain methods of a topical formulation for transdermal delivery of a buffering agent provided herein may be supplemented with methods described in greater detail in the inventor's related applications mentioned above, including PCT/US2018/028017; PCT/US2018/051250; PCT/US2020/038558; PCT/US2022/012330; PCT/US2022/080338; PCT/US2022/080368; and PCT/US2023/012833. The contents of each of which is incorporated by reference in its entirety.

### Transdermal Delivery Topical Formulations

An aspect of the present disclosure is a topical formulation for transdermal delivery of a buffering agent through the skin of a subject. The topical formulation comprising one or more buffering agents, one or more fatty acid esters, one or more alcohols, one or more long-chain fatty acids, one or more oils, one or more poloxamers, one or more phospholipids, polyglyceryl-4 laurate, and water.

An advantage of a transdermal drug delivery route over other types of delivery is that the formulation can provide a controlled release of the agent. Conventional transdermal delivery systems are generally ineffective for use with agents and medications that are large molecules and/or hydrophilic molecules.

There are other advantages to transdermal administration of medicaments. Small molecules can be inactivated or degraded by the stomach or liver. Transdermal administration is not affected by stomach or digestive issues. Further, people can benefit from drugs that are absorbed slowly and regularly. With a transdermal formulation, a medicament can be released in small quantities over a long period of time.

Other advantages are related to dosing. Large doses of agents can cause dose-dependent toxicity in many cases. For example, oral administration of vitamin A can result in hypervitaminosis A. The main problems associated with the vitamin A are its half-life, fast absorption (due to lipophilicity) and its toxicity (due to high loading and frequent dosing). Also, some drugs undergo first-pass metabolism, which prevents their delivery to the desired site of action. Furthermore, many hydrophilic or lipophilic drugs show either poor dissolution or poor absorption on oral administration. With a transdermal formulation, the effective concentration of a buffering agent can be applied at the desired site without painful delivery.

In various embodiments, a topical formulation for transdermal delivery of a buffering agent comprises the components of the below table:

Generic Topical Formulation

| Ingredient | Weight (%) |
|---|---|
| Buffering agents (e.g., sodium bicarbonate | 25%-40%, e.g., |
| (baking soda or sodium hydrogen carbonate), | 30% to 35% |
| tris (tromethamine, trisaminomethane, | |
| 2-amino-2-hydroxymethyl-propane-1,3-diol, or | |
| tris(hydroxymethyl)aminomethane), calcium | |
| carbonate, tripotassium phosphate | |
| (potassium phosphate), sodium carbonate | |
| (disodium carbonate), sodium hydroxide | |
| (sodium oxidanide), potassium bicarbonate | |
| (potassium hydrogen carbonate or potassium acid | |
| carbonate), lysine, potassium carbonate, dipotassium | |
| phosphate (potassium phosphate dibasic or | |
| potassium hydrogen phosphate), disodium phosphate | |
| (sodium phosphate dibasic or disodium hydrogen | |
| phosphate), trisodium phosphate, meglumine | |
| ((2r,3r,4r,5s)-6-(methylamino)hexane-1,2,3,4,5- | |
| pentol or methylglucamine), arginine, | |
| triethanolamine (tea or 2,2′,2″- | |
| nitrilotriethanol), glycine, monosodium | |
| phosphate (sodium dihydrogen phosphate), | |
| monopotassium phosphate (potassium | |
| dihydrogen phosphate), monoethanolamine, | |
| diethanolamine (diolamine or 2-(2- | |
| hydroxyethylamino)ethanol), magnesium | |
| carbonate, and 2-imidazole-1-yl-3- | |
| ethoxycarbonylpropionic acid (IEPA)) | |
| Alcohols (e.g., benzyl alcohol, ethanol, cetyl | 1%-13% in total; |
| alcohol, and propylene glycol | 1% to 6% per |
| | alcohol |
| Fatty acid esters (e.g., isopropyl palmitate and | 6%-10%, e.g., |
| isopropyl myristate) | 7% to 9% |
| Long-chain fatty acids (e.g., linoleic, oleic, | 0.3%-2% in total; |
| stearic acid, linolenic, palmitic, arachidonic, | 0.3% to 0.6% per |
| palmitoleic, myristic, eicosenoic, benehic, | LCFA |
| euricic, and lignoceric acid) | |
| Oils (e.g., safflower oil, macadamia oil, almond | 1%-7% in total; |
| oil, another oil with high linoleic composition, | 1.5% to 3.5% |
| or another oil with high oleic composition) | per oil |
| Poloxamers (e.g., poloxamer 407, poloxamer 188, | 4%-7%, e.g., |
| poloxamer 184, and poloxamer 124) | 5% to 6.5% |
| Phospholipids (e.g., phosphatidylcholine, | 3.5%-4.5% |
| hydrogenated phosphatidylcholine, | |
| phosphatidylserine, phosphatidylethanolamine, | |
| phosphatidylinositol, inositol | |
| phosphatide, and sphingomyelin) | |
| Polyglyceryl-4 laurate | 0.5%-1.5% |
| Water | 30%-42% |
| Optionally, glucose | 0.25%-0.45% |

In the above table, where an ingredient has weight percent that ranges from 5 to 20% and the like, that ingredient may be present in the formulation at any percentage (w/w or w/v) from about 5% to about 20%. The weight percentage may be about 5% to about 20%. The weight percentage may be about 5% to about 6%, about 5% to about 7%, about 5% to about 8%, about 5% to about 9%, about 5% to about 10%, about 5% to about 11%, about 5% to about 12%, about 5% to about 13%, about 5% to about 14%, about 5% to about 15%, about 6% to about 7%, about 6% to about 8%, about 6% to about 9%, about 6% to about 10%, about 6% to about 11%, about 6% to about 12%, about 6% to about 13%, about 6% to about 14%, about 6% to about 15%, about 7% to about 8%, about 7% to about 9%, about 7% to about 10%, about 7% to about 11%, about 7% to about 12%, about 7% to about 13%, about 7% to about 14%, about 7% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 10% to about 16%, about 10% to about 17%, about 10% to about 18%, about 10% to about 19%, about 10% to about 20%, about 11% to about 12%, about 11% to about 13%, about 11% to about 14%, about 11% to about 15%, about 11% to about 16%, about 11% to about 17%, about 11% to about 18%, about 11% to about 19%, about 11% to about 20%, about 12% to about 13%, about 12% to about 14%, about 12% to about 15%, about 12% to about 16%, about 12% to about 17%, about 12% to about 18%, about 12% to about 19%, about 12% to about 20%, about 13% to about 14%, about 13% to about 15%, about 13% to about 16%, about 13% to about 17%, about 13% to about 18%, about 13% to about 19%, about 13% to about 20%, about 14% to about 15%, about 14% to about 16%, about 14% to about 17%, about 14% to about 18%, about 14% to about 19%, about 14% to about 20%, about 15% to about 16%, about 15% to about 17%, about 15% to about 18%, about 15% to about 19%, about 15% to about 20%, about 16% to about 17%, about 16% to about 18%, about 16% to about 19%, about 16% to about 20%, about 17% to about 18%, about 17% to about 19%, about 17% to about 20%, about 18% to about 19%, about 18% to about 20%, or about 19% to about 20%, and any range therebetween. The weight percentage may be about 5%, about 6%, about 7%, about 8%, about 9%, about 10%, about 11%, about 12%, about 13%, about 14%, about 15%, about 16%, about 17%, about 18%, about 19%, or about 20%. The weight percentage may be at least about 5%, about 6%, about 7%, about 8%, about 9%, about 10%, about 11%, about 12%, about 13%, or about 14%. The weight percentage may be at most about 6%, about 7%, about 8%, about 9%, about 10%, about 11%, about 12%, about 13%, about 14%, about 15%, about 16%, about 17%, about 18%, about 19%, or about 20%. Moreover, the weight percentage may be about 5% to about 6%. The weight percentage may be about 5% to about 5.1%, about 5% to about 5.2%, about 5% to about 5.3%, about 5% to about 5.4%, about 5% to about 5.5%, about 5% to about 5.6%, about 5% to about 5.7%, about 5% to about 5.8%, about 5% to about 5.9%, about 5% to about 6%, about 5.1% to about 5.2%, about 5.1% to about 5.3%, about 5.1% to about 5.4%, about 5.1% to about 5.5%, about 5.1% to about 5.6%, about 5.1% to about 5.7%, about 5.1% to about 5.8%, about 5.1% to about 5.9%, about 5.1% to about 6%, about 5.2% to about 5.3%, about 5.2% to about 5.4%, about 5.2% to about 5.5%, about 5.2% to about 5.6%, about 5.2% to about 5.7%, about 5.2% to about 5.8%, about 5.2% to about 5.9%, about 5.2% to about 6%, about 5.3% to about 5.4%, about 5.3% to about 5.5%, about 5.3% to about 5.6%, about 5.3% to about 5.7%, about 5.3% to about 5.8%, about 5.3% to about 5.9%, about 5.3% to about 6%, about 5.4% to about 5.5%, about 5.4% to about 5.6%, about 5.4% to about 5.7%, about 5.4% to about 5.8%, about 5.4% to about 5.9%, about 5.4% to about 6%, about 5.5% to about 5.6%, about 5.5% to about 5.7%, about 5.5% to about 5.8%, about 5.5% to about 5.9%, about 5.5% to about 6%, about 5.6% to about 5.7%, about 5.6% to about 5.8%, about 5.6% to about 5.9%, about 5.6% to about 6%, about 5.7% to about 5.8%, about 5.7% to about 5.9%, about 5.7% to about 6%, about 5.8% to about 5.9%, about 5.8% to about 6%, or about 5.9% to about 6%. The weight percentage may be about 5%, about 5.1%, about 5.2%, about 5.3%, about 5.4%, about 5.5%, about 5.6%, about 5.7%, about 5.8%, about 5.9%, or about 6%. The weight percentage may be at least about 5%, about 5.1%, about 5.2%, about 5.3%, about 5.4%, about 5.5%, about 5.6%, about 5.7%, about 5.8%, or about 5.9%. The weight percentage may be at most about 5.1%, about 5.2%, about 5.3%, about 5.4%, about 5.5%, about 5.6%, about 5.7%, about 5.8%, about 5.9%, or about 6%. Further, the weight percentage may be about 5% to about 5.1%. The weight percentage may be about 5% to about 5.01%, about 5% to about 5.02%, about 5% to about 5.03%, about 5% to about 5.04%, about 5% to about 5.05%, about 5% to about 5.06%, about 5% to about 5.07%, about 5% to about 5.08%, about 5% to about 5.09%, about 5% to about 5.1%, about 5.01% to about 5.02%, about 5.01% to about 5.03%, about 5.01% to about 5.04%, about 5.01% to about 5.05%, about 5.01% to about 5.06%, about 5.01% to about 5.07%, about 5.01% to about 5.08%, about 5.01% to about 5.09%, about 5.01% to about 5.1%, about 5.02% to about 5.03%, about 5.02% to about 5.04%, about 5.02% to about 5.05%, about 5.02% to about 5.06%, about 5.02% to about 5.07%, about 5.02% to about 5.08%, about 5.02% to about 5.09%, about 5.02% to about 5.1%, about 5.03% to about 5.04%, about 5.03% to about 5.05%, about 5.03% to about 5.06%, about 5.03% to about 5.07%, about 5.03% to about 5.08%, about 5.03% to about 5.09%, about 5.03% to about 5.1%, about 5.04% to about 5.05%, about 5.04% to about 5.06%, about 5.04% to about 5.07%, about 5.04% to about 5.08%, about 5.04% to about 5.09%, about 5.04% to about 5.1%, about 5.05% to about 5.06%, about 5.05% to about 5.07%, about 5.05% to about 5.08%, about 5.05% to about 5.09%, about 5.05% to about 5.1%, about 5.06% to about 5.07%, about 5.06% to about 5.08%, about 5.06% to about 5.09%, about 5.06% to about 5.1%, about 5.07% to about 5.08%, about 5.07% to about 5.09%, about 5.07% to about 5.1%, about 5.08% to about 5.09%, about 5.08% to about 5.1%, or about 5.09% to about 5.1%. The weight percentage may be about 5%, about 5.01%, about 5.02%, about 5.03%, about 5.04%, about 5.05%, about 5.06%, about 5.07%, about 5.08%, about 5.09%, or about 5.1%. The weight percentage may be at least about 5%, about 5.01%, about 5.02%, about 5.03%, about 5.04%, about 5.05%, about 5.06%, about 5.07%, about 5.08%, or about 5.09%. The weight percentage may be at most about 5.01%, about 5.02%, about 5.03%, about 5.04%, about 5.05%, about 5.06%, about 5.07%, about 5.08%, about 5.09%, or about 5.1%.

The other ranges recited in the above table include similar ranges and subranges and values within ranges.

In some embodiments, the one or more alcohols are present in an amount from about 1% to about 13% w/w of the topical formulation. In some cases, the one or more alcohols are selected from benzyl alcohol, ethanol, cetyl alcohol, and propylene glycol. When present benzyl alcohol may be in an amount from about 1% to about 2% w/w of the topical formulation; when present ethanol may be in an amount from about 1% to about 2% w/w of the topical formulation; when present cetyl alcohol may be in an amount from about 1.5% to about 2.5% w/w of the topical formulation; and when present propylene glycol may be in an amount from about 4% to about 6% w/w of the topical formulation.

In various embodiments, the one or more fatty acid esters are present in an amount from about 6% to about 10% w/w of the topical formulation.

In additional embodiments, the one or more fatty acid esters are present in an amount from about 7% to about 9% w/w of the topical formulation.

In embodiments, the one or more fatty acid esters is selected from isopropyl palmitate and isopropyl myristate.

In some embodiments, the one or more long-chain fatty acids are present in an amount from about 0.3% to about 2% w/w of the topical formulation.

In various embodiments, the one or more long the long-chain fatty acids are selected from a linoleic, oleic, stearic acid, linolenic, palmitic, arachidonic, palmitoleic, myristic, eicosenoic, benehic, euricic, and lignoceric acid. When present stearic acid may be in an amount from about 0.3% to about 0.5% w/w of the topical formulation and when present oleic acid may be in an amount from about 0.4% to about 0.6% w/w of the topical formulation.

In additional embodiments, the one or more oil are present in an amount from about 1% to about 7% w/w of the topical formulation.

In embodiments, the one or more oils are safflower oil, macadamia oil, almond oil, another oil with high linoleic composition, or another oil with high oleic composition. When present safflower oil may be in an amount from about 1.5% to about 2% w/w of the topical formulation and when present almond oil may be in an amount from about 2.5% to about 3.5% w/w of the topical formulation.

In some embodiments, the one or more poloxamers are in an amount from about 4% to about 7% w/w of the topical formulation.

In various embodiments, the one or more poloxamers are in an amount from about 5% to about 6.5% w/w of the topical formulation.

In additional embodiments, the one or more poloxamers are selected from poloxamer 407, poloxamer 188, poloxamer 184, and poloxamer 124.

In embodiments, the one or more phospholipids are in an amount from about 3.5% to about 4.5% w/w of the topical formulation.

In some embodiments, the one or more phospholipids are selected from phosphatidylcholine, hydrogenated phosphatidylcholine, phosphatidylserine, phosphatidylethanolamine, phosphatidylinositol, inositol phosphatide, and sphingomyelin.

In various embodiments, the polyglyceryl-4 laurate is in an amount from about 0.5% to about 1.5% w/w of the topical formulation.

In additional embodiments, the polyglyceryl-4 laurate is in an amount of about 1% w/w of the topical formulation.

In embodiments, the water is in an amount from about 30% to about 42% w/w of the topical formulation.

In some embodiments, the one or more buffering agents are in an amount from about 25% to about 40% w/w of the topical formulation.

In various embodiments, the one or more buffering agents are in an amount from about 30% to about 35% w/w of the topical formulation.

In additional embodiments, the one or more buffering agents are in an amount of about 33% w/w of the topical formulation.

In embodiments, the one or more buffering agents are selected from sodium bicarbonate (baking soda or sodium hydrogen carbonate), tris (tromethamine, trisaminomethane, 2-amino-2-hydroxymethyl-propane-1,3-diol, or tris(hydroxymethyl)aminomethane), calcium carbonate, tripotassium phosphate (potassium phosphate), sodium carbonate (disodium carbonate), sodium hydroxide (sodium oxidanide), potassium bicarbonate (potassium hydrogen carbonate or potassium acid carbonate), lysine, potassium carbonate, dipotassium phosphate (potassium phosphate dibasic or potassium hydrogen phosphate), disodium phosphate (sodium phosphate dibasic or disodium hydrogen phosphate), trisodium phosphate, meglumine ((2r,3r,4r,5s)-6-(methylamino)hexane-1,2,3,4,5-pentol or methylglucamine), arginine, triethanolamine (tea or 2,2′,2″-nitrilotriethanol), glycine, monosodium phosphate (sodium dihydrogen phosphate), monopotassium phosphate (potassium dihydrogen phosphate), monoethanolamine, diethanolamine (diolamine or 2-(2-hydroxyethylamino)ethanol), magnesium carbonate, and 2-imidazole-1-yl-3-ethoxycarbonylpropionic acid (IEPA).

In some embodiments, the topical formulation comprises about 33% sodium bicarbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate.

In various embodiments, the topical formulation comprises about 33% calcium carbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate.

In additional embodiments, the topical formulation comprises about 33% sodium bicarbonate, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate.

In embodiments, the topical formulation comprises about 33% tris, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate.

In an embodiment, a topical formulation for transdermal delivery of a buffering agent contains a phosphatide in a concentration of at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

In various embodiments, a formulation lacks a natural (e.g., plant or animal derived) lecithin.

In an embodiment, a topical formulation for transdermal delivery of a buffering agent contains a sterol or benzyl alcohol in a concentration of at least 1%, at least 2%, at least 3%, at least 4%, at least 5%, at least 6%, at least 7%, at least 8%, at least 9%, at least 10%, at least 15%, at least 20%, at least 25%, at least 30% or more w/w of the transdermal delivery formulation.

In an embodiment, a topical formulation for transdermal delivery of a buffering agent contains a carbohydrate in a concentration of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

In various embodiments, a topical formulation for transdermal delivery of a buffering agent contains no glucose.

In an embodiment, a topical formulation for transdermal delivery of a buffering agent contains water in a concentration of at least 0.1%, at least 0.2%, at least 0.3%, at least 0.4%, at least 0.5%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

In various embodiments, a formulation lacks a natural (e.g., plant or animal derived) lecithin.

In a further embodiment, a fatty acid is a saturated or an unsaturated fatty acid. In another embodiment, an unsaturated fatty acid is myristoleic acid, palmitoleic acid, sapienic acid, oleic acid, elaidic acid, vaccenic acid, linoleic acid, linoelaidic acid, α-Linolenic acid, arachidonic acid, eicosapentaenoic acid, erucic acid and/or docosahexaenoic acid. In an embodiment, a saturated fatty acid is caprylic acid, capric acid, lauric acid, myristic acid, palmitic acid, stearic acid, arachidic acid, behenic acid, lignoceric acid and/or cerotic acid. In another embodiment, the fatty acid is a dietary fat and include duct fat, lard, tallow, butter, coconut oil, cocoa butter, palm kernel oil, palm oil, cottonseed oil, wheat germ oil, soybean oil, olive oil, corn oil, sunflower oil, macadamia oil, safflower oil, hemp oil and/or canola/rapeseed oil, or another oil with high linoleic composition.

A topical formulation for transdermal delivery of a buffering agent comprises mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize one or more distinct permeation enhancers.

In some embodiments, carotenoids are excluded from the formulations disclosed.

In an aspect, the concentration of phosphatidylcholine in a topical formulation for transdermal delivery of a buffering agent is at least 10%, at least 15%, at least 20%, at least 25%, at least 28.75%, at least 30%, at least 35%, at least 40% or more. In an aspect, the concentration of phosphatidylcholine in a topical formulation for transdermal delivery of a buffering agent is not more than 10%, not more than 15%, not more than 20%, not more than 25%, not more than 28.75%, not more than 30%, not more than 35%, not more than 40% or more. In an aspect, the concentration of phosphatidylcholine in a topical formulation for transdermal delivery of a buffering agent is about 10%, about 15%, about 20%, about 25%, at least 28.75%, about 30%, about 35%, about 40% or more. In an aspect, the concentration of Phosphatidylcholine in a topical formulation for transdermal delivery of a buffering agent is from 10% to 40%, is from 15% to 35%, is from 20% to 30%, is from 25% to 30%, is from 28% to 29%.

In a further embodiment, the concentration of benzyl alcohol in a transdermal formulation is at least 0.25%, at least 0.5%, at least 0.75%, at least 1%, at least 2%, at least 2.5%, at least 3%, at least 4%, at least 5% or more. In an embodiment, the concentration of Benzyl Alcohol in a transdermal formulation is about 0.25%, about 0.5%, about 0.75%, about 1%, about 2%, about 2.5%, about 3%, about 4%, about 5% or more. In another embodiment, the concentration of Benzyl Alcohol in a transdermal formulation is at from 0.25% to 5%; from 0.5% to 4%, from 0.75% to 3%, from 1% to 2.5% or from 0.5% to 2%. In a further embodiment, the concentration of Benzyl Alcohol in a transdermal formulation is no more than 0.25%, no more than 0.5%, no more than 0.75%, no more than 1%, no more than 2%, no more than 2.5%, no more than 3%, no more than 4%, no more than 5%.

In an embodiment, the concentration of deionized water in a transdermal formulation is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5% or more. In an embodiment, the concentration of Deionized Water in a transdermal formulation is about 0.1%, about 0.2%, about 0.3%, about 0.4%, about 0.5%, about 0.6%, about 0.7%, about 0.8%, about 0.9%, about 1%, about 2%, about 3%, about 4%, about 5% or more. In an embodiment, the concentration of deionized water in a transdermal formulation is from 0.1% to 5%, from 0.2% to 4%, from 0.3% to 3%, 0.4%-2%, 0.5% to 1%, from 0.6% to 0.9%, from 0.7% to 0.8%, from 0.4% to 1.5%, from 0.3% to 0.7% or from 0.4% to 0.6%. In an embodiment, the concentration of deionized water in a transdermal formulation is no more than 0.1%, no more than 0.2%, no more than 0.3%, no more than 0.4%, no more than 0.5%, no more than 0.6%, no more than 0.7%, no more than 0.8%, no more than 0.9%, no more than 1%, no more than 2%, no more than 3%, no more than 4%, no more than 5% or more.

In an aspect, the concentration of safflower oil in a topical formulation for transdermal delivery of a buffering agent is at least 1%, at least 5%, at least 7.5%, at least 10%, at least 11%, at least 11.06%, at least 12%, at least 13%, at least 14%, at least 15%, at least 16%, at least 17%, at least 18%, at least 19%, at least 20% or more. In an aspect, the concentration of Safflower oil in a topical formulation for transdermal delivery of a buffering agent is about 1%, about 5%, about 7.5%, about 10%, about 11%, about 11.06%, about 12%, about 13%, about 14%, about 15%, about 16%, about 17%, about 18%, about 19%, about 20% or more. In an aspect, the concentration of Safflower oil in a topical formulation for transdermal delivery of a buffering agent is from 1% to 20%, from 5% to 19%, from 7.5% to 18%, from 10% to 17%, from 11% to 16%, from 11.06%, 12% from 11% to 12%, from 12% to 14%, from 13% to 14%, from 10% to 12%, from 10.5% to 12.5% or from 11% to 11.25%. In an aspect, the concentration of safflower oil in a topical formulation for transdermal delivery of a buffering agent is no more than 1%, no more than 5%, no more than 7.5%, no more than 10%, no more than 11%, no more than 11.06%, no more than 12%, no more than 13%, no more than 14%, no more than 15%, no more than 16%, no more than 17%, no more than 18%, no more than 19%, no more than 20%.

In a further aspect, the concentration of oleic acid in a topical formulation for transdermal delivery of a buffering agent is at least 1%, at least 2%, at least 3%, at least 3.65%, at least 4%, at least 5%, at least 6%, at least 7%, at least 8%, at least 9%, at least 10% or more. In a further aspect, the concentration of oleic acid in a topical formulation for transdermal delivery of a buffering agent is about 1%, about 2%, about 3%, about 3.65%, about 4%, about 5%, about 6%, about 7%, about 8%, about 9%, about 10% or more. In a further aspect, the concentration of Oleic Acid in a topical formulation for transdermal delivery of a buffering agent is no more than 1%, no more than 2%, no more than 3%, no more than 3.65%, no more than 4%, no more than 5%, no more than 6%, no more than 7%, no more than 8%, no more than 9%, no more than 10% or more. In another aspect, the concentration of Oleic Acid in a transdermal formulation is from 1% to 10%, from 2% to 9%, from 2% to 3%, from 3% to 4%, from 3% to 8%, from 4% to 7%, from 5% to 6%, from 2 to 2.5% or from 2.5% to 4%.

In another aspect, the concentration of stearic acid in a transdermal formulation is at least 1%, at least 2%, at least 2.34%, at least 3%, at least 4%, at least 5%, at least 6%, at least 7%, at least 8%, at least 9%, at least 10% or more. In another aspect, the concentration of stearic acid in a transdermal formulation is no more than 1%, no more than 2%, no more than 2.34%, no more than 3%, no more than 4%, no more than 5%, no more than 6%, no more than 7%, no more than 8%, no more than 9%, no more than 10% or more. In another aspect, the concentration of stearic acid in a transdermal formulation is about 1%, about 2%, about 2.34%, about 3%, about 4%, about 5%, about 6%, about 7%, about 8%, about 9%, about 10% or more. In another aspect, the concentration of stearic acid in a transdermal formulation is from 1% to 10%, from 2% to 9%, from 2% to 3%, from 2.34% to 2.5%, from 3% to 8%, from 4% to 7%, from 5% to 6% or from 1.5% to 2.5%.

In an aspect, the concentration of isopropyl palmitate in a transdermal formulation is at least 10%, at least 20%, at least 25%, at least 30%, at least 40%, at least 45%, at least 50%, at least 55%, at least 60%, at least 65%, at least 70%, at least 75% or more. In an aspect, the concentration of isopropyl palmitate in a transdermal formulation is about 10%, about 20%, about 25%, about 30%, about 40%, about 45%, about 50%, about 55%, about 60%, about 65%, about 70%, about 75% or more. In an aspect, the concentration of isopropyl palmitate in a transdermal formulation is no more than 10%, no more than 20%, no more than 25%, no more than 30%, no more than 40%, no more than 45%, no more than 50%, no more than 55%, no more than 60%, no more than 65%, no more than 70%, no more than 75% or more. In an aspect, the concentration of isopropyl palmitate in a transdermal formulation is from 10% to 75%, from 20% to 70%, from 25% to 65%, from 30% to 60%, from 40% to 55%, from 45% to 50%, from 40% to 60%, from 45% to 55% or from 47% to 53%.

In an aspect, the concentration of poloxamer 407 in a topical formulation for transdermal delivery of a buffering agent is at least 10%, at least 15%, at least 20%, at least 25%, at least 28.75%, at least 30%, at least 35%, at least 40% or more. In an aspect, the concentration of poloxamer 407 in a topical formulation for transdermal delivery of a buffering agent is not more than 10%, not more than 15%, not more than 20%, not more than 25%, not more than 28.75%, not more than 30%, not more than 35%, not more than 40% or more. In an aspect, the concentration of poloxamer 407 in a topical formulation for transdermal delivery of a buffering agent is about 10%, about 15%, about 20%, about 25%, at least 28.75%, about 30%, about 35%, about 40% or more. In an aspect, the concentration of poloxamer 407 in a topical formulation for transdermal delivery of a buffering agent is from 10% to 40%, is from 15% to 35%, is from 20% to 30%, is from 25% to 30%, is from 28% to 29%.

In another aspect, the formulation includes glucose. The concentration of glucose in a topical formulation for transdermal delivery of a buffering agent can be, for example, at least 1%, at least 2%, at least 2.5%, at least 3%, at least 4%, at least 5%, at least 6%, at least 7%, at least 8%, at least 9% or more. In another aspect, the concentration of glucose in a topical formulation for transdermal delivery of a buffering agent is about 1%, about 2%, about 2.5%, about 3%, about 4%, about 5%, about 6%, about 7%, about 8%, about 9% or more. In another aspect, the concentration of glucose in a topical formulation for transdermal delivery of a buffering agent is no more than 1%, no more than 2%, no more than 2.5%, no more than 3%, no more than 4%, no more than 5%, no more than 6%, no more than 7%, no more than 8%, no more than 9% or more. In another aspect, the concentration of glucose in a topical formulation for transdermal delivery of a buffering agent is from 1% to 10%, is from 2% to 9%, is from 2.5% to 5%, is from 2% to 3%, is from 3% to 8%, if from 4% to 7%, if from 5% to 6%, is from 2% to 4%, is from 1.5% to 3.5%. In various embodiments, a topical formulation for transdermal delivery of a buffering agent contains no glucose.

A topical formulation for transdermal delivery of a buffering agent can include mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers.

In some embodiments, a topical formulation for transdermal delivery of a buffering agent comprises phosphatidylcholine in amount less than 12% w/w or 18% w/w of the formulation. In some embodiments, the topical formulation for transdermal delivery of a buffering agent comprises a phospholipid in amount less than 12% w/w or 18% w/w of the formulation. In some embodiments, the topical formulation for transdermal delivery of a buffering agent comprises a mixture of tridecane and undecane in amount less than 2% w/w, 5% w/w, or 8% w/w of the formulation. In some embodiments, the formulation comprises Cetiol Ultimate® in an amount less than about 2% w/w, 5% w/w, or 10% w/w, or an equivalent mixture of tridecane and undecane. In some embodiments, the topical formulation for transdermal delivery of a buffering agent comprises cetyl alcohol in amount less than 2% w/w, 5% w/w, or 8% w/w of the formulation. In some embodiments, the topical formulation for transdermal delivery of a buffering agent comprises benzyl alcohol in an amount less than about 2% w/w, 5% w/w, or 8% w/w. In some embodiments, the topical formulation for transdermal delivery of a buffering agent comprises stearic acid in an amount less than 2% w/w, 5% w/w, or 8% w/w of the formulation.

For topical administration, and in particular transdermal administration, a topical formulation for transdermal delivery of a buffering agent will comprise penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. In some embodiments, suitable penetrants include those that are described in the above-referenced US2009/0053290, WO2014/209910, and WO2017/127834. In addition to transdermal delivery formulations with penetrants, transdermal delivery can be affected by mechanically disrupting the surface of the skin to encourage penetration, or simply by supplying the formulation applied to the skin under an occlusive patch.

Alternatively, the topical formulation for transdermal delivery of a buffering agent comprises a completion component as well as one or more electrolytes sufficient to impart viscosity and viscoelasticity, one or more surfactants and an alcohol. The completion component can be a polar liquid, a non-polar liquid or an amphiphilic substance. The penetrant may further comprise a keratinolytic agent effective to reduce thiol linkages, disrupt hydrogen bonding and/or effect keratin lysis and/or a cell penetrating peptide (sometimes referred to as a skin-penetrating peptide) and/or a permeation enhancer.

Suitable gelling components also include isopropyl palmitate, ethyl laurate, ethyl myristate and isopropyl myristate. In some embodiments, a topical formulation for transdermal delivery of a buffering agent comprises a gelling agent in an amount less than 5% w/w of a transdermal delivery formulation. Certain hydrocarbons, such as cyclopentane, cyclooctane, trans-decalin, trans-pinane, n-pentane, n-hexane, n-hexadecane may also be used. In some embodiments, the topical formulation for transdermal delivery of a buffering agent comprises a mixture of xanthan gum, sclerotium gum, pullulan, or a combination thereof in an amount less than 2% w/w, 5% w/w, or 10% w/w of the formulation. In some embodiments, a topical formulation for transdermal delivery of a buffering agent comprises Siligel™ in an amount from about 1 to about 5% w/w or from about 5 to about 15% w/w, or an equivalent mixture of xanthan gum, sclerotium gum, and pullulan. In some embodiments, a topical formulation for transdermal delivery of a buffering agent comprises a mixture of caprylic triglycerides and capric triglycerides in amount less than 2% w/w, 8% w/w, or 10% w/w of the formulation. In some embodiments, a topical formulation for transdermal delivery of a buffering agent comprises Myritol® 312 in an amount from about 0.5 to about 10% w/w, or an equivalent mixture of caprylic triglycerides and capric triglycerides.

An additional component in a topical formulation for transdermal delivery of a buffering agent of the disclosure is an alcohol. Benzyl alcohol, cetyl alcohol, and ethanol are illustrated in the Examples. In particular, derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 3% w/w, 4% w/w, 5% w/w, 6% w/w, 7% w/w, 8% w/w, 9% w/w, or 10% w/w, and other intermediate weight percentages are included. Due to the aromatic group present in a topical formulation for transdermal delivery of a buffering agent such as benzyl alcohol, the molecule has a polar end (the alcohol end) and a non-polar end (the benzene end). This enables the agent to dissolve a wider variety of topical formulation for transdermal delivery of a buffering agent components.

In some embodiments, as noted above, the performance of a topical formulation for transdermal delivery of a buffering agent is further improved by including a nonionic detergent and polar gelling agent or including a powdered surfactant. In both aqueous and anhydrous forms of the composition, detergents, typically nonionic detergents are added. In general, the nonionic detergent should be present in an amount from about 1% w/w to about 30% w/w of a transdermal delivery formulation. Typically, in the compositions wherein a topical formulation for transdermal delivery of a buffering agent is topped off with a polar or aqueous solution containing detergent, the amount of detergent is relatively low—e.g., 2-25% w/w, or 5-15% w/w or 7-12% w/w of a transdermal delivery formulation. However, in compositions that are essentially anhydrous and are topped-off by powdered detergent, relatively higher percentages are usually used—e.g., 20-60% w/w.

In some embodiments, a topical formulation for transdermal delivery of a buffering agent further comprises a detergent portion in an amount from about 1 to about 70% w/w or from about 1 to about 60% w/w of a transdermal delivery formulation. In some embodiments, the nonionic detergent provides suitable handling properties whereby the formulations are gel-like or creams at room temperature. To exert this effect, the detergent, typically a poloxamer, is present in an amount from about 2 to about 12% w/w of a transdermal delivery formulation, preferably from about 5 to about 25% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as the non-ionic surfactant Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, a topical formulation for transdermal delivery of a buffering agent also contains surfactant, typically, nonionic surfactant at 2-25% w/w of a topical formulation for transdermal delivery of a buffering agent along with a polar solvent wherein the polar solvent is present in an amount at least in molar excess of the nonionic surfactant. In these embodiments, typically, the composition comprises the above-referenced amounts of a topical formulation for transdermal delivery of a buffering agent and benzyl alcohol along with a sufficient amount of a polar solution, typically an aqueous solution or polyethylene glycol solution that itself contains 10%-40% of surfactant, typically nonionic surfactant to bring the composition to 100%.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by Sigma-Aldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w-15% w/w, and again includes intermediate percentages such as 5% w/w, 7% w/w, 10% w/w, 12% w/w, and the like. In some embodiments, the detergent portion comprises a nonionic surfactant in an amount from about 1 to about 30% w/w of the formulation, and a polar solvent in an amount less than 5% w/w of the formulation. In some embodiments, the nonionic surfactant is a poloxamer and the polar solvent is water, an alcohol, or a combination thereof. In some embodiments, the detergent portion comprises poloxamer, propylene glycol, glycerin, ethanol, 50% w/v sodium hydroxide solution, or a combination thereof. In some embodiments, the detergent portion comprises glycerin in an amount less than 3% w/w of the formulation.

In the presence of a polar gelling agent, such as water, glycerol, ethylene glycol or formamide, a micellular structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature unless otherwise stated. The concentration of hydroxypropylcellulose may range from about 1% w/w to about 2% w/w of the composition. Other gelling agents are known in the art and can be used in place of, or in addition to hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Additional components that can also be included in a topical formulation for transdermal delivery of a buffering agent are fatty acids, terpenes, lipids, and cationic, and anionic detergents. In some embodiments, a topical formulation for transdermal delivery of a buffering agent further comprises tranexamic acid in an amount less than 2% w/w, 5% w/w, or 10% w/w of the formulation. In some embodiments, a topical formulation for transdermal delivery of a buffering agent further comprises a polar solvent in an amount less than 2% w/w, 5% w/w, 10% w/w, or 20% w/w of the transdermal delivery formulation. In some embodiments, a topical formulation for transdermal delivery of a buffering agent further comprises a humectant, an emulsifier, an emollient, or a combination thereof. In some cases, the humectant is propylene glycol. In some cases, the emulsifier is polyglyceryl-4-laurate, cetyl alcohol or Durosoft® PK-SG. In some cases, the emollient is derived from almond oil. In some embodiments, a topical formulation for transdermal delivery of a buffering agent further comprises almond oil in an amount less than about 5% w/w. In some embodiments, a formulation further comprises a mixture of thermoplastic polyurethane and polycarbonate in an amount less than about 5% w/w. In some embodiments, a topical formulation for transdermal delivery of a buffering agent further comprises phosphatidylethanolamine in an amount less than about 5% w/w. In some embodiments, a topical formulation for transdermal delivery of a buffering agent further comprises an inositol phosphatide in an amount less than about 5% w/w.

Other solvents and related compounds that can be used in some embodiments include acetamide and derivatives, acetone, n-alkanes (chain length from 7 to 16), alkanols, diols, short chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefosse, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C₃-C₆ triols or their mixtures and a polar lipid compound selected from C₁₆ or C₁₈ monounsaturated alcohol, C₁₆ or C₁₈ branched saturated alcohol and their mixtures, propylene glycol, sorbitan monolaurate sold as Span® 20 by Sigma-Aldrich, squalene, triacetin, trichloroethanol, trifluoroethanol, trimethylene glycol and xylene.

Fatty alcohols, fatty acids, fatty esters, are bilayer fluidizers that can be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monooleate, sorbitan trilaurate, sorbitan trioleate, sucrose coconut fatty ester mixtures, sucrose monolaurate, sucrose monooleate, tetradecyl N.N-dimethylamino acetate. Examples of suitable fatty acid—include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include a-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′-dihydroxypropyl glycerol).

Examples of completing agents that can be used in some embodiments include β- and γ-cyclodextrin complexes, hydroxypropyl methylcellulose (e.g., Carbopol® 934), liposomes, naphthalene diamide diimide, and naphthalene diester diimide.

One or more anti-oxidants can be included, such as vitamin C, vitamin E, proanthocyanidin and a-lipoic acid typically in concentrations of 0.1%-2.5% w/w.

In some applications, it is desirable to adjust the pH of a topical formulation for transdermal delivery of a buffering agent to assist in permeation or to adjust the nature of the target compounds in the subject. In some instances, the pH is adjusted to a level of pH 9-11 or 10-11 which can be done by providing appropriate buffers or simply adjusting the pH with base.

A topical formulation for transdermal delivery of a buffering agent can include other components that act as excipients or serve purposes other than for muscle performance and recovery. For example, preservatives like antioxidants e.g., ascorbic acid or α-lipoic acid and antibacterial agents may be included. Other components apart from therapeutically active ingredients and components that are the primary effectors of dermal penetration may include those provided for aesthetic purposes such as menthol or other aromatics, and components that affect the physical state of the composition such as emulsifiers, for example, Durosoft®. Typically, these ingredients are present in very small percentages of the compositions. It is understood that these latter ancillary agents are neither therapeutically ingredients nor are they components that are primarily responsible for penetration of the skin. The components that primarily effect skin penetration have been detailed as described above. However, some of these substances have some capability for effecting skin penetration. See, for example, Kunta, J. R. et al, J. Pharm. Sci. (1997) 86:1369-1373, describing penetration properties of menthol.

The application method is determined by the nature of the treatment but may be less critical than the nature of the formulation itself. If the application is to a skin area, it may be helpful in some instances to prepare the skin by cleansing or exfoliation. In some instances, it is helpful to adjust the pH of the skin area prior to application of a topical formulation for transdermal delivery of a buffering agent itself. The application of a topical formulation for transdermal delivery of a buffering agent may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a topical formulation for transdermal delivery of a buffering agent and allowing the penetration to occur so as to restore the skin barrier. A convenient way to do this is to apply a composition comprising linoleic acid which effectively closes the entrance pathways that were provided by the penetrants of the present disclosure. This application, too, is done by straightforward smearing onto the skin area or can be applied more precisely in measured amounts.

In addition to the compositions and formulations of the present disclosure per se, the methods can employ a subsequent treatment with linoleic acid. As transdermal treatments generally open up the skin barrier, which is, indeed, their purpose, it is useful to seal the area of application after the treatment is finished. Thus, treatment with a topical formulation for transdermal delivery of a buffering agent may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow the active component to pass through the epidermis to the dermis at least, and, if systemic administration is achieved, through the dermis itself.

Additional therapeutic agents can be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol can be included in an amount ranging from about 0.1% w/w to about 1.0% w/w.

The compositions containing anesthetics are useful for temporary relief of pain and itching associated with minor burns, cuts, scrapes, skin irritations, inflammation, and rashes due to soaps, detergents or cosmetics, or, as noted above, pain associated with removal of fat deposits.

In some particular embodiments it is desirable to adjust the pH of a topical formulation for transdermal delivery of a buffering agent and the pH is adjusted to a level of pH 9-11 or 10-11, which can be done by providing appropriate buffers or simply adjusting the pH with base. In other embodiments, it is desirable to adjust the pH of a topical formulation for transdermal delivery of a buffering agent to a level of pH 4-6, which can be done by providing appropriate buffers or simply adjusting the pH with an acid.

In some applications a formulation for transdermal delivery may, for example, comprise: Aveeno®, for example in an amount from about 10 to about 95% w/w; from about 20 to about 85% w/w, from about 20 to about −75% w/w, from about 20 to about 50% w/w.

The formulation described in this specification may also comprise more than one therapeutic compound as desired for the particular indication being treated, preferably those with complementary activities that do not adversely affect the other proteins. A topical formulation for transdermal delivery of a buffering agent to be used for in vivo administration can be sterile. This can be accomplished, for instance, without limitation, by filtration through sterile filtration membranes, prior to, or following, preparation of a topical formulation for transdermal delivery of a buffering agent or other methods known in the art, including without limitation, pasteurization.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

In certain embodiments, kits can comprise, without limitation, one or more cream, lotion, ointment or the like comprising one or more formulations described herein. In various embodiments, the kit can comprise formulation components for transdermal, topical, or subcutaneous administration, formulated to be administered as an emulsion coated patch. In all of these embodiments and others, the kits can contain one or more lotion, ointment, cream, patch, or the like in accordance with any of the foregoing, wherein each patch contains a single unit dose for administration to a subject.

Imaging components can optionally be included, and the packaging also can include written or web-accessible instructions for using a transdermal delivery formulation. A container can include, for example, a vial, bottle, patch, syringe, pre-filled syringe, tube or any of a variety of formats well known in the art for multi-dispenser packaging.

In an aspect, the concentration of poloxamer 407 in a topical formulation for transdermal delivery of a buffering agent is at least 10%, at least 15%, at least 20%, at least 25%, at least 28.75%, at least 30%, at least 35%, at least 40% or more. In an aspect, the concentration of poloxamer 407 in a topical formulation for transdermal delivery of a buffering agent is not more than 10%, not more than 15%, not more than 20%, not more than 25%, not more than 28.75%, not more than 30%, not more than 35%, not more than 40% or more. In an aspect, the concentration of poloxamer 407 in a topical formulation for transdermal delivery of a buffering agent is about 10%, about 15%, about 20%, about 25%, at least 28.75%, about 30%, about 35%, about 40% or more. In an aspect, the concentration of poloxamer 407 in a topical formulation for transdermal delivery of a buffering agent is from 10% to 40%, is from 15% to 35%, is from 20% to 30%, is from 25% to 30%, is from 28% to 29%.

A topical formulation for transdermal delivery of a buffering agent can include mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers.

Certain components or ingredients of a topical formulation for transdermal delivery of a buffering agent provided herein may be supplemented with components described in greater detail in the inventor's related applications mentioned above, including PCT/US2018/028017; PCT/US2018/051250; PCT/US2020/038558; PCT/US2022/012330; PCT/US2022/080338; PCT/US2022/080368; and PCT/US2023/012833. The contents of each of which is incorporated by reference in its entirety.

### Methods for Treating a Liver Disease

Methods for treating, preventing or ameliorating a disease, disorder, a condition, or a symptom thereof or a condition related thereto are provided herein using a topical formulation for transdermal delivery of a buffering agent for transdermal delivery described herein. The methods provided herein may comprise or consist of topically administering one or more of a topical formulation for transdermal delivery of a buffering agent described herein to skin of a subject in need thereof. Preferred, but non-limiting embodiments are directed to methods for treating, preventing, inhibiting or ameliorating a disease, disorder, a condition, or a symptom described herein.

An aspect of the present disclosure is a method for treating a liver disease or reducing the risk of acquiring a liver disease. The method comprising administering to a subject in need thereof a topical formulation for transdermal delivery of a buffering agent. The topical formulation comprises a penetrant portion and a therapeutically effective amount of the buffering agent. In this aspect, the penetrant portion comprises: a phospholipid, a fatty acid ester formed from a low molecular weight alcohol, and a long-chain fatty acids, and, optionally, one or more of a viscosity-improving agents, a penetration enhancer, an oil, and an emulsifier, and the buffering agent comprises one or more of sodium bicarbonate (baking soda or sodium hydrogen carbonate), tris (tromethamine, trisaminomethane, 2-amino-2-hydroxymethyl-propane-1,3-diol, or tris(hydroxymethyl)aminomethane), calcium carbonate, tripotassium phosphate (potassium phosphate), sodium carbonate (disodium carbonate), sodium hydroxide (sodium oxidanide), potassium bicarbonate (potassium hydrogen carbonate or potassium acid carbonate), lysine, potassium carbonate, dipotassium phosphate (potassium phosphate dibasic or potassium hydrogen phosphate), disodium phosphate (sodium phosphate dibasic or disodium hydrogen phosphate), trisodium phosphate, meglumine ((2r,3r,4r,5s)-6-(methylamino)hexane-1,2,3,4,5-pentol or methylglucamine), arginine, triethanolamine (tea or 2,2′,2″-nitrilotriethanol), glycine, monosodium phosphate (sodium dihydrogen phosphate), monopotassium phosphate (potassium dihydrogen phosphate), monoethanolamine, diethanolamine (diolamine or 2-(2-hydroxyethylamino)ethanol), magnesium carbonate, and 2-imidazole-1-yl-3-ethoxycarbonylpropionic acid (IEPA).

In embodiments, the phospholipid is selected from phosphatidylcholine, hydrogenated phosphatidylcholine, phosphatidylserine, phosphatidylethanolamine, phosphatidylinositol, inositolphosphatide, and sphingomyelin. In some cases, the phospholipid is phosphatidylcholine.

In some embodiments, the penetrant portion comprises two or more phospholipids.

In various embodiments, the phospholipid is in an amount from about 3% to about 15% w/w of the topical formulation.

In additional embodiments, the low molecular weight alcohol is selected from isopropanol, methanol, ethanol, butanol, glycerol, cetyl alcohol.

In embodiments, the low molecular weight alcohol is isopropanol.

In some embodiments, the fatty acid ester is selected from isopropyl palmitate, isopropyl myristate, isopropyl linoleate, isopropyl oleate, ethyl laurate, and ethyl myristate. In some cases, the fatty acid ester is isopropyl palmitate.

In various embodiments, the fatty acid ester is in an amount from about 5% to about 20% w/w of the topical formulation.

In additional embodiments, the fatty acid ester is in an amount from about 7% to about 9% w/w of the topical formulation.

In embodiments, the long-chain fatty acid is selected from a linoleic, oleic, stearic acid, linolenic, palmitic, arachidonic, palmitoleic, myristic, eicosenoic, benehic, euricic, and lignoceric acid. In some cases, the long-chain fatty acid is linoleic acid or oleic acid and/or the long-chain fatty acid is stearic acid.

In some embodiments, the long-chain fatty acid is in an amount from about 0.1% to about 2% w/w of the topical formulation.

In various embodiments, the oil is safflower oil, macadamia oil, almond oil, another oil with high linoleic composition, or another oil with high oleic composition.

In additional embodiments, the oil is in an amount from about 1% to about 7% w/w of the topical formulation.

In embodiments, the oil is almond oil and in an amount from about 2.5% to about 3.5% w/w of the topical formulation.

In some embodiments, the oil is safflower oil and in an amount from about 1.5% to about 2.5% w/w of the topical formulation.

In various embodiments, the penetrant portion comprises a viscosity-improving agent.

In additional embodiments, the viscosity-improving agent is a poloxamer. In some cases, the poloxamer is selected from poloxamer 407, poloxamer 188, poloxamer 184, and poloxamer 124. In some cases, the poloxamer is in an amount from about 4% to about 7% w/w of the topical formulation.

In embodiments, the viscosity-improving agent is polyglyceryl-4 laurate. In some cases, the polyglyceryl-4 laurate is in an amount from about 0.5% to about 2% w/w of the topical formulation. In some cases, the polyglyceryl-4 laurate is in an amount of about 1% w/w of the topical formulation.

In some embodiments, the penetrant portion comprises a penetration enhancer.

In various embodiments, the penetration enhancer is an alcohol or a terpene. In some cases, the penetration enhancer is an alcohol selected from benzyl alcohol, ethanol, propylene glycol, cetyl alcohol, and polyethylene glycol. In some cases, the penetration enhancer comprises two or more alcohols.

In additional embodiments, the penetration enhancer further acts as a preservative.

In embodiments, the penetrant portion comprises two or more penetration enhancers.

In some embodiments, the penetration enhancer is in an amount from about 1% to about 20% w/w of the topical formulation.

In various embodiments, the penetration enhancer is in an amount from about 6% to about 15% w/w of the topical formulation.

In additional embodiments, the penetrant portion comprises at least one penetration enhancer and at least one viscosity-improving agent.

In embodiments, the penetrant portion comprises an emulsifier.

In some embodiments, the emulsifier is selected from polyglyceryl-4-laurate, polyglyceryl-4-oleate, span 60, cetyl alcohol, and polyglyceryl-3-oleate.

In various embodiments, the penetrant portion comprises two or more emulsifiers.

In additional embodiments, the emulsifier is in an amount from about 0.5 to about 10% w/w of the topical formulation.

In embodiments, the penetrant portion comprises at least one emulsifier and at least one viscosity-improving agent.

In some embodiments, the penetrant portion comprises at least one emulsifier and at least one penetration enhancer.

In various embodiments, the penetrant portion comprises at least one emulsifier, at least one viscosity-improving agent, and at least one penetration enhancer.

In additional embodiments, the topical formulation further comprises water. In some cases, the water is in an amount from about 30% water to about 50% w/w of the topical formulation. In some cases, the water is in an amount from about 33% water to about 42% w/w of the topical formulation.

In embodiments, the topical formulation further comprises glucose. In some cases, the glucose is in an amount from about 0.25% to about 0.5% w/w of the topical formulation. In some cases, the glucose is in an amount of about 0.55% w/w of the topical formulation.

In some embodiments, the buffering agent is in an amount from about 25% to about 50% w/w of the topical formulation. In some cases, the buffering agent is in an amount from about 30% to about 35% w/w of the topical formulation. In some cases, the buffering agent is in an amount of about 33% w/w of the topical formulation.

In various embodiments, the formulation is in the form of a cream, lotion, ointment, or the like.

In additional embodiments, the topical formulation increases pH systemically in the subject.

In embodiments, the topical formulation increases the subject's urine pH.

In some embodiments, the liver disease is a non-alcoholic fatty liver disease (NAFLD).

In various embodiments, the liver disease is a non-alcoholic steatohepatitis (NASH).

In additional embodiments, the liver disease is cirrhosis, hepatocellular carcinoma, hepatitis, alcoholic liver disease, hepatic ischemia/reperfusion injury from liver transplant or resection, Budd-Chiari syndrome, primary biliary cholangitis, primary sclerosing cholangitis, progressive familial intrahepatic cholestasis, and Wilson disease.

In embodiments, the subject has cirrhosis or the liver and/or hepatocellular carcinoma (HCC).

In some embodiments, the subject has insulin resistance and/or diabetes, e.g., Type 1 or Type 2 diabetes.

In various embodiments, the subject partakes in a high-fat diet and/or partakes in a high-calorie diet.

In additional embodiments, the subject has a higher than recommended serum cholesterol and/or has a higher than recommended serum triglyceride levels.

In embodiments, the subject has an inflammatory disease and/or chronic inflammation.

In some embodiments, the subject has increased expression of inflammatory cytokines, e.g., Interleukin 1 beta (IL-1β), Tumor Necrosis Factor alpha (TNF-α), Interleukin 6 (IL-6), and Transforming Growth Factor beta (TGF-β).

In various embodiments, the subject has an abnormal gut microbiota.

A subject in need thereof is selected for having a liver disease or at risk for having a liver disease. In some cases, the subject in need thereof is not administered a topical formulation of the present disclosure for transdermal delivery of a buffering agent before being selected for having a liver disease or at risk for having a liver disease. In other words, the subject had not previously been administered a topical formulation of the present disclosure. Thus, the subject in need is specifically selected to be administered ministered a topical formulation of the present disclosure due a diagnosis of having a liver disease or at risk for having a liver disease.

In some embodiments, the topical formulation comprises about 33% sodium bicarbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate.

In various embodiments, the topical formulation comprises about 33% calcium carbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate.

In additional embodiments, the topical formulation comprises about 33% sodium bicarbonate, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate.

In embodiments, the topical formulation comprises about 33% tris, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate.

A topical formulation for transdermal delivery of a buffering agent provided herein can be topically administered in any form. For administration for the treatment of skin conditions a sufficient amount of the topical composition can be applied onto a desired area and surrounding skin, for example, in an amount sufficient to cover a desired skin surface. A topical formulation for transdermal delivery of a buffering agent can be applied to any skin surface, including for example, facial skin, and the skin of the hands, neck, chest and/or scalp.

In applying a topical formulation for transdermal delivery of a buffering agent of the present disclosure, a topical formulation for transdermal delivery of a buffering agent itself is simply placed on the skin and spread across the surface and/or massaged to aid in penetration. The amount of topical formulation for transdermal delivery of a buffering agent used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of a topical formulation for transdermal delivery of a buffering agent to the skin and prevents distortion of a topical formulation for transdermal delivery of a buffering agent by evaporation in some cases. The composition may be applied to the skin using standard procedures for application such as a brush, a syringe, a gauze pad, a dropper, or any convenient applicator. More complex application methods, including the use of delivery devices, may also be used, but are not required. In an alternative to administering topically to intact skin, the surface of the skin may also be disrupted mechanically by the use of spring systems, laser powered systems, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

In an alternative to administering topically to intact skin, the surface of the skin may also be disrupted mechanically by the use of spring systems, laser powered systems, use of iontophoresis, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed transdermal delivery formulations that penetrate intact skin may be applied using occlusive patches, such as those in the form of micro-patches. External reservoirs of the formulations for extended administration may also be employed.

Accordingly, in certain embodiments alternative methods of administering one or more therapeutic compounds or agents (e.g., medicaments) through intact skin are provided. As nonlimiting examples, these alternative methods might be selected from the following lists: on basis of working mechanism, spring systems, laser powered, energy-propelled, Lorentz force, gas/air propelled, shock wave (including ultrasound), on basis of type of load, liquid, powder, projectile, on basis of drug delivery mechanism, nano-patches, sandpaper (microdermabrasion), iontophoresis enabled, microneedles, on basis of site of delivery, intradermal, intramuscular, and subcutaneous injection. Other suitable delivery mechanisms include, without limitation, microneedle drug delivery, such as 3M Systems, Glide SDI (pushes drug as opposed to “firing” drug), MIT low pressure injectors, micropatches (single use particle insertion device), microelectro mechanical systems (MEMS), dermoelectroporation devices (DEP), transderm ionto system (DEP), TTS transdermal therapeutic systems, membrane-moderated systems (drug reservoir totally encapsulated in a shallow compartment), adhesive diffusion-controlled system (drug reservoir in a compartment fabricated from drug-impermeable metallic plastic backing), matrix dispersion type system (drug reservoir formed by homogeneously dispersing drug solids in a hydrophilic or lipophilic polymer matrix molder into medicated disc), and microreservoir system (combination of reservoir and matrix dispersion-type drug delivery system).

The application method is determined by the nature of the treatment but may be less critical than the nature of a topical formulation for transdermal delivery of a buffering agent itself. If the application is to a skin area, it may be helpful in some instances to prepare the skin by cleansing or exfoliation. In some instances, it is helpful to adjust the pH of the skin area prior to application of the formulation itself. The application of a topical formulation for transdermal delivery of a buffering agent may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a topical formulation for transdermal delivery of a buffering agent and allowing the penetration to occur so as to restore the skin barrier. A convenient way to do this is to apply a composition comprising linoleic acid which effectively closes the entrance pathways that were provided by the penetrants of the present disclosure. This application, too, is done by straightforward smearing onto the skin area or can be applied more precisely in measured amounts.

A topical formulation for transdermal delivery of a buffering agent can be applied in a single, one-time application, once a week, once a bi-week, once a month, or from one to twelve times daily, for a period of time sufficient to alleviate a condition, disease, disorder, symptoms, for example, for a period of time of one week, from 1 to 12 weeks or more, from 1 to 6 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day to hourly if needed. The presently described formulations can be topically administered once or more per day for a period of time from 1 week to 4 weeks, from 1 week to 2 weeks, for 1 week, for 2 weeks, for 3 weeks, or for 4 weeks or more. In some instances, it may also be desirable to continue treatment indefinitely, for example, to inhibit recurring inflammation. A suitable administration for a topical formulation for transdermal delivery of a buffering agent comprising a skin cream, lotion, or ointment, for example is once, twice, three, four times daily, or hourly if needed.

As described above, if desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A topical formulation for transdermal delivery of a buffering agent in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the topical formulation for transdermal delivery of a buffering agent may be prepared. Single dose, unit dose, and once-daily disposable containers of the topical formulation for transdermal delivery of a buffering agent are also provided.

The present topical formulation for transdermal delivery of a buffering agent remains stable in storage for periods including up to about 5 years, from about 3 months to about 5 years, from about 3 months to about 4 years, from about 3 months to about 3 years, and alternately any time period from about 6 months to about 3 years.

A topical formulation for transdermal delivery of a buffering agent described herein remains stable for up to at least 3 years at a temperature of less than or equal to 40° C. In an embodiment, the presently described topical formulation for transdermal delivery of a buffering agent remains stable for at least 2 years at a temperature of less than or equal to 40° C. In an embodiment, the presently described topical formulation for transdermal delivery of a buffering agent remains stable for at least 3 years at a temperature of less than or equal to 40° C. and at a humidity of up to 75% RH, for at least 2 years at a temperature of less than or equal to 40° C. and at a humidity of up to 75% RH, or for at least 3 years at a temperature of less than or equal to 30° C. and at a humidity of up to 75% RH. In a further embodiment, the presently described topical formulation for transdermal delivery of a buffering agent in accordance with the subject matter described herein remains stable for an extended period of time when packaged in a multi-use container such as a bottle dispenser or the like and exhibits equal to or even greater stability when packaged in a single-use package.

In another aspect, the topical formulation for transdermal delivery of a buffering agent of certain embodiments comprises a daily dose of particular buffering agent (e.g., sodium bicarbonate, sodium carbonate, magnesium carbonate, potassium carbonate, potassium bicarbonate, TRIS, Lysine, IEPA, and other buffering agents as disclosed herein). A daily dose for topical or transdermal administration of a topical formulation for transdermal delivery of a buffering agent depends on the compound and animal and may be easily determined by the skilled artisan, a suitable amount is about 1 mg/kg to about 5 g/kg, and more typically the daily dose is about 10 mg/kg to about 5 g/kg, about 25 mg/kg to about 2000 mg/kg, about 50 mg/kg to about 2000 mg/kg, about 25 mg/kg to about 1000 mg/kg, about 50 mg/kg to about 1000 mg/kg, about 100 mg/kg to about 700 mg/kg, about 100 mg/kg to about 500 mg/kg, about 150 mg/kg to about 500 mg/kg, about 150 mg/kg to about 400 mg/kg, about 200 mg/kg to about 500 mg/kg, about 200 mg/kg to about 450 mg/kg, about 200 mg/kg to about 400 mg/kg, about 250 mg/kg to about 450 mg/kg, about 250 mg/kg to about 400 mg/kg, about 250 mg/kg to about 350 mg/kg, and about 275 mg/kg to about 325 mg/kg.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A topical formulation for transdermal delivery of a buffering agent in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the topical formulation for transdermal delivery of a buffering agent may be prepared. Single dose, unit dose, and once-daily disposable containers of the topical formulation for transdermal delivery of a buffering agent are also provided.

Alternatively, a suitable dose for topical or transdermal administration of each of one or more particular buffering agent (e.g., sodium bicarbonate, sodium carbonate, magnesium carbonate, potassium carbonate, potassium bicarbonate, TRIS, Lysine, IEPA, and others agents as disclosed herein) for subject is at least about 100 mg, at least about 500 mg, at least about 1 g, at least about 5 g, at least about 10 g, at least about 15 g, at least about 16 g, at least about 17 g, at least about 18 g, at least about 19 g, at least about 20 g, at least about 21 g, at least about 22 g, at least about 23 g, at least about 24 g, at least about 25 g, at least about 26 g, at least about 27 g, at least about 28 g, at least about 29 g, at least about 30 g, at least about 35 g, at least about 40 g, at least about 45 g, at least about 50 g, at least about 60 g, at least about 75 g, at least about 100 g, at least about 200 g, at least about 500 g, or at least about 1.0 kg. This dose may be administered daily, twice a day, three times a day, four times a day, five times a day, or more than five times a day.

Aspects of the present specification disclose that the symptoms associated with a disease or disorder described herein are reduced following application of a topical formulation for transdermal delivery of a buffering agent by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced following application of a topical formulation for transdermal delivery of a buffering agent by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

Aspects of the present specification disclose that the symptoms associated with a disease or disorder described herein are reduced following administration of a topical formulation for transdermal delivery of a buffering agent of the present disclosure by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A topical formulation for transdermal delivery of a buffering agent as described herein can be used in the manufacture of medicaments and for the treatment of humans and other animals by administration in accordance with conventional procedures.

Dosing can be single dosage or cumulative (serial dosing), and can be readily determined by one skilled in the art. A topical formulation for transdermal delivery of a buffering agent of the present disclosure may be administered once, twice, three, four, five, six, seven, eight, nine, ten, eleven, twelve, thirteen, fourteen, fifteen, sixteen, seventeen, eighteen, nineteen, twenty or more times to a subject. For instance, treatment of a disease may comprise a one-time administration of an effective dose of a topical formulation for transdermal delivery of a buffering agent as disclosed herein. Alternatively, treatment of a disease may comprise multiple administrations of an effective dose of a topical formulation for transdermal delivery of a buffering agent as carried out over a range of time periods, such as, e.g., once daily, twice daily, thrice daily, once every few days, or once weekly. The timing of administration can vary from individual to individual, depending upon such factors as the severity of an individual's symptoms. For example, an effective dose of a topical formulation for transdermal delivery of a buffering agent as disclosed herein can be administered to an individual once daily for an indefinite period of time, or until the individual no longer requires therapy. A person of ordinary skill in the art will recognize that the condition of the individual can be monitored throughout the course of treatment and that the effective amount of a topical formulation for transdermal delivery of a buffering agent disclosed herein that is administered can be adjusted accordingly. In one embodiment, a topical formulation for transdermal delivery of a buffering agent as disclosed herein is capable of decreasing the time to resolve the symptoms of a disease, including in an individual suffering from a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

In a further embodiment, a topical formulation for transdermal delivery of a buffering agent and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a topical formulation for transdermal delivery of a buffering agent is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a topical formulation for transdermal delivery of a buffering agent disclosed herein reduces or alleviates symptoms (e.g., muscle pain or cramping) of an ailment such as muscle cramping or soreness in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a topical formulation for transdermal delivery of a buffering agent disclosed herein reduces or alleviates symptoms of an ailment in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a topical formulation for transdermal delivery of a buffering agent disclosed herein reduces or alleviates symptoms of an ailment in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A topical formulation for transdermal delivery of a buffering agent disclosed herein may comprise a topical formulation for transdermal delivery of a buffering agent in a therapeutically effective amount. As used herein, the term “effective amount” is synonymous with “therapeutically effective amount”, “effective dose”, or “therapeutically effective dose” and when used in reference to reducing or alleviate symptoms of an ailment in an individual refers to the minimum dose of a therapeutic disclosed herein necessary to achieve the desired therapeutic effect and includes a dose sufficient to reduce or alleviates symptoms of an ailment in an individual. The effectiveness of a topical formulation for transdermal delivery of a buffering agent disclosed herein capable of reducing or alleviating symptoms of an ailment in an individual can be determined by observing an improvement in an individual based upon one or more clinical symptoms, and/or physiological indicators associated with improvements in muscle performance, reduced soreness and/or overall health. Maintenance or a reduction of symptoms of an ailment can also be subjective to a patient. The effectiveness of a topical formulation for transdermal delivery of a buffering agent disclosed herein in an individual can be determined by observing an improvement in an individual based upon one or more clinical symptoms, and/or physiological indicators associated with signs/symptoms, muscle performance and general health. The effectiveness of a topical formulation for transdermal delivery of a buffering agent disclosed herein is also capable of prolonging the life of an individual as compared to the same individual if the topical formulation for transdermal delivery of a buffering agent is not administered. The effectiveness of the topical formulation for transdermal delivery of a buffering agent disclosed herein is also capable of enhancing the quality of life of an individual as compared to the same individual if the topical formulation for transdermal delivery of a buffering agent is not administered.

The appropriate effective amount of a topical formulation for transdermal delivery of a buffering agent disclosed herein to be administered to an individual can be determined by a person of ordinary skill in the art by taking into account factors, including, without limitation, an improvement in the individual based upon one or more clinical symptoms, and/or physiological indicators associated with improvements in muscle performance, reduced soreness and/or overall health, the particular characteristics, history and risk factors of the patient, such as, e.g., age, weight, general health and the like, or any combination thereof. Additionally, where repeated administration of a topical formulation for transdermal delivery of a buffering agent is used, an effective amount of a topical formulation for transdermal delivery of a buffering agent will further depend upon factors, including, without limitation, the frequency of administration, the half-life of the transdermal delivery formulation, or any combination thereof. It is known by a person of ordinary skill in the art that an effective amount of a topical formulation for transdermal delivery of a buffering agent disclosed herein can be extrapolated from in vitro assays and in vivo administration studies using animal models prior to administration to humans or animals.

Wide variations in the necessary effective amount are to be expected in view of the differing efficiencies of the various routes of administration. For instance, oral administration of a topical formulation for transdermal delivery of a buffering agent disclosed herein generally would be expected to require higher dosage levels than administration by inhalation. Similarly, systemic administration of a topical formulation for transdermal delivery of a buffering agent disclosed herein would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a therapeutic disclosed herein that is administered can be adjusted accordingly.

Aspects of the present specification disclose, in part, a reduction or alleviation of symptoms of an ailment such as muscle cramping or soreness in an individual. As used herein, the term “treating,” refers to reduction or alleviation of muscle soreness or cramping as well as improved muscle performance and recovery. For example, the term “treating” can mean reduction or alleviation of symptoms in an individual by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with an ailment such as muscle cramping and soreness are well known and can be determined by a person of ordinary skill in the art by using commonly known testing means. Those of skill in the art will know the appropriate symptoms or indicators associated with health and muscle performance and will know how to determine if an individual is a candidate for treatment as disclosed herein.

In an embodiment, a first topical formulation for transdermal delivery of a buffering agent is administered to an individual and at a later date, a second topical formulation for transdermal delivery of a buffering agent is administered to the same individual. In an embodiment, a first topical formulation for transdermal delivery of a buffering agent is administered to an individual at the same time as a second topical formulation for transdermal delivery of a buffering agent is administered to the individual.

A topical formulation for transdermal delivery of a buffering agent as disclosed herein is administered to an individual. An individual is typically a human being, but can be an animal, including, but not limited to, dogs, cats, birds, cattle, horses, sheep, goats, reptiles and other animals, whether domesticated or not.

Certain components or ingredients of a topical formulation for transdermal delivery of a buffering agent provided herein may be supplemented with components described in greater detail in the inventor's related applications mentioned above, including PCT/US2018/028017; PCT/US2018/051250; PCT/US2020/038558; PCT/US2022/012330; PCT/US2022/080338; PCT/US2022/080368; and PCT/US2023/012833. The contents of each of which is incorporated by reference in its entirety.

### Combination Therapies.

In some embodiments, the subject with a liver disease is administered a topical formulation for transdermal delivery of a buffering agent and is additionally administered a therapeutic for treating the liver disease or a related disorder. In some cases, the therapeutic for treating the liver disease or a related disorder is administered before, contemporary with, or after the topical formulation. In some cases, the therapeutic is administered orally, topically, enterally, parenterally, by intravenous injection or infusion, by intraperitoneal injection, by intramuscular injection, or by subcutaneous injection. In some cases, the therapeutic is a liquid, a suspension, a gel, a geltab, a semisolid, a tablet, a sachet, a lozenge, a pill, or a capsule. In some cases, the therapeutic is a drug relating to glycemic control (e.g., a sodium-glucose cotransporter-2 (SGLT2) inhibitor), a drug for lipid metabolism (e.g., a fibrate), and/or a NASH drug that has failed in a clinical trial due to lack of efficacy (e.g., elafibranor, cenicriviroc, remoglifozin, Obeticholic, Resmetirom, and Nalmefene).

In embodiments, the subject is additionally administered a therapeutic for treating the liver disease or a related disorder, with the therapeutic being included in the topical formulation. In some cases, transdermal delivery of the therapeutic via the topical formulation provides systemic administration of the therapeutic. In some cases, the therapeutic is a drug relating to glycemic control (e.g., a sodium-glucose cotransporter-2 (SGLT2) inhibitor), a drug for lipid metabolism (e.g., a fibrate), and/or a NASH drug that has failed in a clinical trial due to lack of efficacy (e.g., elafibranor, cenicriviroc, remoglifozin, Obeticholic, Resmetirom, and Nalmefene).

Various transdermal formulations of the present disclosure are able to successfully deliver (at relatively high bioavailability) a range of actives that are either entirely insoluble, at least partially, or majority insoluble. This ability to deliver insoluble actives is contrary to the commonly-held belief that actives generally need to be solubilized for transdermal drug delivery.

There are a number of pharmacologically active medicaments which are poorly soluble in in aqueous media and/or acidic solutions which have proved challenging to deliver to a subject either transdermally or orally, due to the inability of the drug to be absorbed by the small intestine, or inability of the medicament to be solubilized in a transdermal formulation and penetrate the skin. In general, it has previously been thought that pharmacologically active medicaments must be wholly or partially dissolved in an aqueous solution in order to be absorbed by a subject and achieve a reasonably high bioavailability in a subject. In the case of orally administered medicaments, it is thought a drug must be wholly or partially soluble in an aqueous medium in order dissolved in the stomach and absorbed by the small intestine. In the case of transdermally administered drugs, it is thought that a medicament must be wholly or mostly soluble in the formulation in order to penetrate the skin and achieve a high or relatively high bioavailability in a subject.

Such medicaments which are poorly soluble in aqueous medium are drugs which fall into Class 2 and Class 4 of the Biopharmaceutical Classification System (BCS). The BCS is a scientific framework which predicts in vivo drug performance via in vitro measurements of solubility and permeation. Solubility is the extent a drug can dissolve in gastrointestinal (GI) fluid and permeation is the extent solubilized drug can pass across membranes of cells lining the GI tract. Medicaments have a high solubility according to BCS if less than 250 mL of an aqueous media (pH between 1-7.5) dissolves the highest API prescription dose. Class 2 drugs and Class 4 drugs have low solubilities as they do not meet this solubility criteria. Class 2 drugs have high permeability whereas class 4 drugs have low permeability.

Without wishing to be bound by theory, the mechanism of action in which the insoluble class 2 or class 4 medicaments are delivered at a high or reasonably high bioavailability transdermally may include one or more ingredients in the transdermal formulations of present embodiments partially breaking down one or more layers of dermal tissue (e.g., stratum corneum, stratum lucidum, stratum granulosum, stratum spinosum, stratum basale) permitting the medicament to penetrate the dermis, and enter the targeted tissue at a high local concentration, providing a high or reasonably high bioavailability in the targeted tissue. In other embodiments, the mechanism of action may include one or more ingredients in the transdermal formulations of present embodiments partially breaking down one or more layers of dermal tissue (e.g., stratum corneum, stratum lucidum, stratum granulosum, stratum spinosum, stratum basale) in order to permitting the medicament to penetrate the dermis and enter the blood stream as to delivery an insoluble class 2 or class 4 medicaments systemically while bypassing the GI track.

### Manufacturing Formulations.

A topical formulation for transdermal delivery of a buffering agent of the disclosure may be prepared in a number of ways. Typically, the components of a topical formulation for transdermal delivery of a buffering agent are simply mixed together in the required amounts. However, it is also desirable in some instances to, for example, carry out dissolution of a carbonate salt and then add a separate preparation containing the components aiding the delivery of the carbonate salts in the form of a carrier. The concentrations of these components in the carrier, then, will be somewhat higher than the concentrations required in a final transdermal delivery formulation. Thus, the buffering agent may first be dissolved in water and then added to a carrier comprising an alcohol, a topical formulation for transdermal delivery of a buffering agent and optionally a combination of a nonionic surfactant and polar gelling agent, or of ionic detergent. Alternatively, some subset of these components can first be mixed and then “topped off’ with the remaining components either simultaneously or sequentially. The precise manner of preparing a topical formulation for transdermal delivery of a buffering agent will depend on the choice of carbonates and the percentages of the remaining components that are desirable with respect to that carbonate salt. In some embodiments, the water is in an amount from about 10 to about 85% w/w, from about 15 to about 50% w/w, or from about 15 to about 45% w/w of the formulation.

The topical formulation for transdermal delivery of a buffering agent is a multi-component mixture, whereby the particular concentrations of the penetration enhancers are informed in part by the molecular mass of the buffering agent, or the buffering agent and the therapeutic agent to be transported. A topical formulation for transdermal delivery of a buffering agent enables the buffering agent and/or therapeutic agent to become bio-available to the target site within minutes of topical administration. A topical formulation for transdermal delivery of a buffering agent permits the use of minimal concentrations of therapeutic agents, as little as 1/1000th of concentrations required of alternative processes, while enabling bioactivity and positive clinical outcomes simultaneously.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

As used in the specification and claims, the singular forms “a”, “an” and “the” include plural references unless the context clearly dictates otherwise.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

As used herein, the term “about” a number refers to that number plus or minus 10% of that number and/or within one standard deviation (plus or minus) from that number. The term “about” a range refers to that range minus 10% of its lowest value and plus 10% of its greatest value and that range minus one standard deviation its lowest value and plus one standard deviation of its greatest value.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “medicament,” “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

In an embodiment, a “pharmaceutical composition” is intended to include the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

In an embodiment, “an effective amount” refers to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effect may, without necessarily being therapeutic, also be a cosmetic effect, in particular for treatment for disorders of the skin or muscles.

In an embodiment, as used herein, the terms “treating,” “treatment” and the like are used herein to mean obtaining a desired pharmacologic and/or physiologic effect. The effect may be prophylactic in terms of completely or partially preventing a disorder or sign or symptom thereof, including reducing the risk of acquiring the disease or disorder, and/or may be therapeutic in terms of amelioration of the symptoms of the disease or infection, or a partial or complete cure for a disorder and/or adverse effect attributable to the disorder. By reducing the risk of acquiring the disease may either preventing in the traditional use of the word or may lengthen the time in which the subject may acquire the disease; as such, reducing the risk may be interpreted as delaying the onset of the disease and/or delaying the time until symptoms from the disease manifest.

The term “bioavailability” refers to the fraction of an administered dose of unchanged drug that reaches the systemic circulation. For example, when a medication is administered intravenously, its bioavailability is 100%. However, when a medication is administered via other routes (such as orally), its bioavailability generally decreases due to incomplete absorption and first-pass metabolism. Bioavailability is one of the essential tools in pharmacokinetics, as bioavailability must be considered when calculating dosages for non-intravenous routes of administration.

Additionally, the use of particular formulations can disrupt the balance of electrolytes and cations, including those such as the Na/K ratio. For example, the administration of formulations containing calcium carbonate can reduce the amount of sodium or other ions which can decrease the potential for reaching a hyponatremic state. Also, the use of calcium carbonate can also increase the serum levels of calcium which can reduce the amount of calcium leeched from the body by high sodium concentrations.

The formulations and methods of use provided herein take these complexities of electrolyte balance into account. One approach utilized herein in making formulations that avoid electrolyte imbalance and cation overload is to use non-metal buffers or buffers without counterions. Suitable buffering agents for these embodiments include Lysine (free base), TRIS, and IEPA.

For transdermal topical administration in particular for agents other than buffer, a suitable formulation typically involves a penetrant that enhances penetration of the skin and is, in some embodiments, composed of chemical permeation enhancers (CPEs). In some cases, it can also include peptides designed to penetrate cells i.e., cell penetrating peptides (CPPs) also known as skin penetrating peptides (SPPs). The formulation can be applied for example in the form of topical cream, lotion, ointment or the like, as described herein. Many known and useful compounds and the like can be found in Remington's Pharmaceutical Sciences (13^{th} Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

For purposes herein, a formulation, a formulation for transdermal delivery and a topical formulation for transdermal delivery of a buffering agent are each a formulation for transdermal delivery, including, the transdermal delivery of an active ingredient for the treatment of a syndrome and or a disease in an individual.

The section headings used herein are for organizational purposes only and are not to be construed as limiting the subject matter described.

### REFERENCES

Lambrecht J, Tacke F. Controversies and Opportunities in the Use of Inflammatory Markers for Diagnosis or Risk Prediction in Fatty Liver Disease. Front Immunol. 2021; 11:634409. Published 2021 Feb. 9. doi:10.3389/fimmu.2020.634409
Kuchuk O, Tuccitto A, Citterio D, et al. pH regulators to target the tumor immune microenvironment in human hepatocellular carcinoma. Oncoimmunology. 2018; 7(7):e1445452. Published 2018 Mar. 26. doi:10.1080/2162402X.2018.1445452
Younossi Z M, Blissett D, Blissett R, et al. The economic and clinical burden of nonalcoholic fatty liver disease in the United States and Europe. Hepatology. 2016; 64(5):1577-1586. doi:10.1002/hep.28785
Parthasarathy, G., Revelo, X. and Malhi, H. Pathogenesis of Nonalcoholic Steatohepatitis: An Overview. Hepatol Commun, 2020; 4: 478-492. https://doi.org/10.1002/hep4.1479
Miyake T, Yoshida S, Yamamoto S, et al. Low Urine pH Is Associated with Non-alcoholic Fatty Liver Disease: A Community-based Cross-sectional Study. Intern Med. 2018; 57(19):2799-2805. doi:10.2169/internalmedicine.0167-17
Scheiner B, Lindner G, Reiberger T, et al. Acid-base disorders in liver disease. J Hepatol. 2017; 67(5):1062-1073. doi:10.1016/j.jhep.2017.06.023
Erra Diaz F, Dantas E, Geffner J. Unravelling the Interplay between Extracellular Acidosis and Immune Cells. Mediators Inflamm. 2018; 2018:1218297. Published 2018 Dec. 30. doi:10.1155/2018/1218297
Ioannou G N. The Role of Cholesterol in the Pathogenesis of NASH. Trends Endocrinol Metab. 2016; 27(2):84-95. doi:10.1016/j.tem.2015.11.008 https://www.cedars-sinai.org/health-library/diseases-and-conditions/n/non-alcoholic-steatohepatitis-nash.html
Grabherr F, Grander C, Effenberger M, Adolph T E, Tilg H. Gut Dysfunction and Non-alcoholic Fatty Liver Disease. Front Endocrinol (Lausanne). 2019; 10:611. Published 2019 Sep. 6. doi:10.3389/fendo.2019.00611
Ferro D, Baratta F, Pastori D, Cocomello N, Colantoni A, Angelico F, Del Ben M. New Insights into the Pathogenesis of Non-Alcoholic Fatty Liver Disease: Gut-Derived Lipopolysaccharides and Oxidative Stress. Nutrients. 2020; 12(9):2762. https://doi.org/10.3390/nu12092762
Zhang X Q, Xu C F, Yu C H, Chen W X, Li Y M. Role of endoplasmic reticulum stress in the pathogenesis of nonalcoholic fatty liver disease. World J Gastroenterol. 2014; 20(7):1768-1776. doi:10.3748/wjg.v20.i7.1768
Fazakerley D J, Krycer J R, Kearney A L, Hocking S L, James D E. Muscle and adipose tissue insulin resistance: malady without mechanism?. J Lipid Res. 2019; 60(10):1720-1732. doi:10.1194/jlr.R087510
Caldwell S, Ikura Y, Dias D, et al. Hepatocellular ballooning in NASH. J Hepatol. 2010; 53(4):719-723. doi:10.1016/j.jhep.2010.04.031
Gillies R J, Pilot C, Marunaka Y, Fais S. Targeting acidity in cancer and diabetes. Biochim Biophys Acta Rev Cancer. 2019; 1871(2):273-280. doi:10.1016/j.bbcan.2019.01.003
Souto G, Donapetry C, Calvino J, Adeva M M. Metabolic acidosis-induced insulin resistance and cardiovascular risk. Metab Syndr Relat Disord. 2011; 9(4):247-253. doi:10.1089/met.2010.0108
Baldini N, Avnet S. The Effects of Systemic and Local Acidosis on Insulin Resistance and Signaling. Int J Mol Sci. 2018; 20(1):126. Published 2018 Dec. 30. doi:10.3390/ijms20010126
DeFronzo R A, Beckles A D. Glucose intolerance following chronic metabolic acidosis in man. Am J Physiol. 1979; 236(4):E328-E334. doi:10.1 152/ajpendo.1979.236.4.E328
Bellasi A, Di Micco L, Santoro D, et al. Correction of metabolic acidosis improves insulin resistance in chronic kidney disease. BMC Nephrol. 2016; 17(1):158. Published 2016 Oct. 22. doi:10.1186/s12882-016-0372-x
Mak R H. Effect of metabolic acidosis on insulin action and secretion in uremia. Kidney Int. 1998; 54(2):603-607. doi:10.1046/j.1523-1755.1998.00023.x
Pavlov V A, Wang H, Czura C J, Friedman S G, Tracey K J. The cholinergic anti-inflammatory pathway: a missing link in neuroimmunomodulation. Mol Med. 2003; 9(5-8):125-134.
Yang L, Roh Y S, Song J, et al. Transforming growth factor beta signaling in hepatocytes participates in steatohepatitis through regulation of cell death and lipid metabolism in mice. Hepatology. 2014; 59(2):483-495. doi:10.1002/hep.26698
Cai J, Zhang X J, Li H. The Role of Innate Immune Cells in Nonalcoholic Steatohepatitis. Hepatology. 2019; 70(3):1026-1037. doi:10.1002/hep.30506
Chen Z, Yu R, Xiong Y, Du F, Zhu S. A vicious circle between insulin resistance and inflammation in nonalcoholic fatty liver disease [published correction appears in Lipids Health Dis. 2018 Feb. 23; 17 (1):33]. Lipids Health Dis. 2017; 16(1):203. Published 2017 Oct. 16. doi:10.1186/s12944-017-0572-9
Ding W, Duan Y, Qu Z, et al. Acidic Microenvironment Aggravates the Severity of Hepatic Ischemia/Reperfusion Injury by Modulating M1-Polarization Through Regulating PPAR-7 Signal. Front Immunol. 2021; 12:697362. Published 2021 Jun. 21. doi:10.3389/fimmu.2021.697362
Wu X, Dong L, Lin X, Li J. Relevance of the NLRP3 Inflammasome in the Pathogenesis of Chronic Liver Disease. Front Immunol. 2017; 8:1728. Published 2017 Dec. 12. doi:10.3389/fimmu.2017.01728
Michalick L, Kuebler W M. TRPV4—A Missing Link Between Mechanosensation and Immunity. Front Immunol. 2020; 11:413. Published 2020 Mar. 10. doi:10.3389/fimmu.2020.00413
Seth R K, Das S, Dattaroy D, et al. TRPV4 activation of endothelial nitric oxide synthase resists nonalcoholic fatty liver disease by blocking CYP2E1-mediated redox toxicity. Free Radic Biol Med. 2017; 102:260-273. doi:10.1016/j.freeradbiomed.2016.11.047
Lan Z, Chen L, Feng J, et al. Mechanosensitive TRPV4 is required for crystal-induced inflammation. Ann Rheum Dis. 2021; 80(12):1604-1614. doi:10.1136/annrheumdis-2021-220295
Rajamaki K, Nordstram T, Nurmi K, et al. Extracellular acidosis is a novel danger signal alerting innate immunity via the NLRP3 inflammasome. J Biol Chem. 2013; 288(19):13410-13419. doi:10.1074/jbc.M112.426254
Dong L, Krewson E A, Yang L V. Acidosis Activates Endoplasmic Reticulum Stress Pathways through GPR4 in Human Vascular Endothelial Cells. Int J Mol Sci. 2017; 18(2):278. Published 2017 Jan. 27. doi:10.3390/ijms18020278
Mirea A M, Tack C J, Chavakis T, Joosten L A B, Toonen E J M. IL-1 Family Cytokine Pathways Underlying NAFLD: Towards New Treatment Strategies. Trends Mol Med. 2018; 24(5):458-471. doi:10.1016/j.molmed.2018.03.005
Ray S C, Baban B, Tucker M A, et al. Oral NaHCO3 Activates a Splenic Anti-Inflammatory Pathway: Evidence That Cholinergic Signals Are Transmitted via Mesothelial Cells. J Immunol. 2018; 200(10):3568-3586. doi:10.4049/jimmunol.1701605
Zhang A, Marsh K, Rastogi R, Kron I L, Yang Z. Bicarbonate treatment before reperfusion attenuates myocardial ischemia/reperfusion injury by inhibition of spleen-mediated inflammatory response. Circulation. 2021; 144:A9769
Bataller R, Brenner D A. Liver fibrosis [published correction appears in J Clin Invest. 2005 April; 115(4):1100]. J Clin Invest. 2005; 115(2):209-218. doi:10.1172/JC124282
Fu J, Du H, Zhang X, Xu X. Pharmacological Inhibition of Transient Receptor Potential Vanilloid 4 (TRPV4) Channel Alleviates Carbon Tetrachloride-Induced Liver Fibrosis in Mice. J Nippon Med Sch. 2019; 86(5):258-262. doi:10.1272/jnms.JNMS.2019_86-407
Wen Y, Jeong S, Xia Q, Kong X. Role of Osteopontin in Liver Diseases. Int J Biol Sci. 2016; 12(9):1121-1128. Published 2016 Aug. 6. doi:10.7150/ijbs.16445
Dutta B, Goswami R, Rahaman S O. TRPV4 Plays a Role in Matrix Stiffness-Induced Macrophage Polarization. Front Immunol. 2020; 11:570195. Published 2020 Dec. 14. doi:10.3389/fimmu.2020.570195
Song J, Ge Z, Yang X, et al. Hepatic stellate cells activated by acidic tumor microenvironment promote the metastasis of hepatocellular carcinoma via osteopontin. Cancer Lett. 2015; 356(2 Pt B):713-720. doi:10.1016/j.canlet.2014.10.021
Drenth J P H, Schattenberg J M. The nonalcoholic steatohepatitis (NASH) drug development graveyard: established hurdles and planning for future success. Expert Opin Investig Drugs. 2020; 29(12):1365-1375. doi:10.1080/13543784.2020.1839888

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1: Transdermal Formulations of the Present Disclosure Provide Systemic Administration of Active Agents

In various embodiments, a topical formulation for transdermal delivery of a buffering agent comprises the components of the below table:

Generic Topical Formulation

| Ingredient | Weight (%) |
|---|---|
| Buffering agents (e.g., sodium bicarbonate | 25%-40%, e.g., |
| (baking soda or sodium hydrogen carbonate), | 30% to 35% |
| tris (tromethamine, trisaminomethane, | |
| 2-amino-2-hydroxymethyl-propane-1,3-diol, or | |
| tris(hydroxymethyl)aminomethane), calcium | |
| carbonate, tripotassium phosphate (potassium | |
| phosphate), sodium carbonate (disodium | |
| carbonate), sodium hydroxide (sodium | |
| oxidanide), potassium bicarbonate | |
| (potassium hydrogen carbonate or potassium | |
| acid carbonate), lysine, potassium carbonate, | |
| dipotassium phosphate (potassium phosphate | |
| dibasic or potassium hydrogen phosphate), | |
| disodium phosphate (sodium phosphate dibasic | |
| or disodium hydrogen phosphate), | |
| trisodium phosphate, meglumine ((2r,3r,4r,5s)-6- | |
| (methylamino)hexane-1,2,3,4,5-pentol or | |
| methylglucamine), arginine, triethanolamine (tea or | |
| 2,2′,2″-nitrilotriethanol), glycine, | |
| monosodium phosphate (sodium dihydrogen | |
| phosphate), monopotassium phosphate (potassium | |
| dihydrogen phosphate), monoethanolamine, | |
| diethanolamine (diolamine or 2-(2- | |
| hydroxyethylamino)ethanol), magnesium carbonate, | |
| and 2-imidazole-1-yl-3-ethoxycarbonylpropionic | |
| acid (IEPA)) | |
| Alcohols (e.g., benzyl alcohol, ethanol, | 1%-13% in total; |
| cetyl alcohol, and propylene glycol | 1% to 6% per |
| | alcohol |
| Fatty acid esters (e.g., isopropyl palmitate | 6%-10%, e.g., |
| and isopropyl myristate) | 7% to 9% |
| Long-chain fatty acids (e.g., linoleic, oleic, stearic | 0.3%-2% in total; |
| acid, linolenic, palmitic, arachidonic, palmitoleic, | 0.3% to 0.6% per |
| myristic, eicosenoic, benehic, euricic, and | LCFA |
| lignoceric acid) | |
| Oils (e.g., safflower oil, macadamia oil, almond oil, | 1%-7% in total; |
| another oil with high linoleic composition, or another | 1.5% to 3.5% |
| oil with high oleic composition) | per oil |
| Poloxamers (e.g., poloxamer 407, poloxamer 188, | 4%-7%, e.g., |
| poloxamer 184, and poloxamer 124) | 5% to 6.5% |
| Phospholipids (e.g., phosphatidylcholine, | 3.5%-4.5% |
| hydrogenated phosphatidylcholine, phosphatidylserine, | |
| phosphatidylethanolamine, phosphatidylinositol, | |
| inositol phosphatide, and sphingomyelin) | |
| Polyglyceryl-4 laurate | 0.5%-1.5% |
| Water | 30%-42% |
| Optionally, glucose | 0.25%-0.45% |

In the above table, where an ingredient has weight percent that ranges from 5 to 20% and the like, that ingredient may be present in the formulation at any percentage (w/w or w/v) from about 5% to about 20%. The weight percentage may be about 5% to about 20%. The weight percentage may be about 5% to about 6%, about 5% to about 7%, about 5% to about 8%, about 5% to about 9%, about 5% to about 10%, about 5% to about 11%, about 5% to about 12%, about 5% to about 13%, about 5% to about 14%, about 5% to about 15%, about 6% to about 7%, about 6% to about 8%, about 6% to about 9%, about 6% to about 10%, about 6% to about 11%, about 6% to about 12%, about 6% to about 13%, about 6% to about 14%, about 6% to about 15%, about 7% to about 8%, about 7% to about 9%, about 7% to about 10%, about 7% to about 11%, about 7% to about 12%, about 7% to about 13%, about 7% to about 14%, about 7% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 10% to about 16%, about 10% to about 17%, about 10% to about 18%, about 10% to about 19%, about 10% to about 20%, about 11% to about 12%, about 11% to about 13%, about 11% to about 14%, about 11% to about 15%, about 11% to about 16%, about 11% to about 17%, about 11% to about 18%, about 11% to about 19%, about 11% to about 20%, about 12% to about 13%, about 12% to about 14%, about 12% to about 15%, about 12% to about 16%, about 12% to about 17%, about 12% to about 18%, about 12% to about 19%, about 12% to about 20%, about 13% to about 14%, about 13% to about 15%, about 13% to about 16%, about 13% to about 17%, about 13% to about 18%, about 13% to about 19%, about 13% to about 20%, about 14% to about 15%, about 14% to about 16%, about 14% to about 17%, about 14% to about 18%, about 14% to about 19%, about 14% to about 20%, about 15% to about 16%, about 15% to about 17%, about 15% to about 18%, about 15% to about 19%, about 15% to about 20%, about 16% to about 17%, about 16% to about 18%, about 16% to about 19%, about 16% to about 20%, about 17% to about 18%, about 17% to about 19%, about 17% to about 20%, about 18% to about 19%, about 18% to about 20%, or about 19% to about 20%, and any range therebetween. The weight percentage may be about 5%, about 6%, about 7%, about 8%, about 9%, about 10%, about 11%, about 12%, about 13%, about 14%, about 15%, about 16%, about 17%, about 18%, about 19%, or about 20%. The weight percentage may be at least about 5%, about 6%, about 7%, about 8%, about 9%, about 10%, about 11%, about 12%, about 13%, or about 14%. The weight percentage may be at most about 6%, about 7%, about 8%, about 9%, about 10%, about 11%, about 12%, about 13%, about 14%, about 15%, about 16%, about 17%, about 18%, about 19%, or about 20%. Moreover, the weight percentage may be about 5% to about 6%. The weight percentage may be about 5% to about 5.1%, about 5% to about 5.2%, about 5% to about 5.3%, about 5% to about 5.4%, about 5% to about 5.5%, about 5% to about 5.6%, about 5% to about 5.7%, about 5% to about 5.8%, about 5% to about 5.9%, about 5% to about 6%, about 5.1% to about 5.2%, about 5.1% to about 5.3%, about 5.1% to about 5.4%, about 5.1% to about 5.5%, about 5.1% to about 5.6%, about 5.1% to about 5.7%, about 5.1% to about 5.8%, about 5.1% to about 5.9%, about 5.1% to about 6%, about 5.2% to about 5.3%, about 5.2% to about 5.4%, about 5.2% to about 5.5%, about 5.2% to about 5.6%, about 5.2% to about 5.7%, about 5.2% to about 5.8%, about 5.2% to about 5.9%, about 5.2% to about 6%, about 5.3% to about 5.4%, about 5.3% to about 5.5%, about 5.3% to about 5.6%, about 5.3% to about 5.7%, about 5.3% to about 5.8%, about 5.3% to about 5.9%, about 5.3% to about 6%, about 5.4% to about 5.5%, about 5.4% to about 5.6%, about 5.4% to about 5.7%, about 5.4% to about 5.8%, about 5.4% to about 5.9%, about 5.4% to about 6%, about 5.5% to about 5.6%, about 5.5% to about 5.7%, about 5.5% to about 5.8%, about 5.5% to about 5.9%, about 5.5% to about 6%, about 5.6% to about 5.7%, about 5.6% to about 5.8%, about 5.6% to about 5.9%, about 5.6% to about 6%, about 5.7% to about 5.8%, about 5.7% to about 5.9%, about 5.7% to about 6%, about 5.8% to about 5.9%, about 5.8% to about 6%, or about 5.9% to about 6%. The weight percentage may be about 5%, about 5.1%, about 5.2%, about 5.3%, about 5.4%, about 5.5%, about 5.6%, about 5.7%, about 5.8%, about 5.9%, or about 6%. The weight percentage may be at least about 5%, about 5.1%, about 5.2%, about 5.3%, about 5.4%, about 5.5%, about 5.6%, about 5.7%, about 5.8%, or about 5.9%. The weight percentage may be at most about 5.1%, about 5.2%, about 5.3%, about 5.4%, about 5.5%, about 5.6%, about 5.7%, about 5.8%, about 5.9%, or about 6%. Further, the weight percentage may be about 5% to about 5.1%. The weight percentage may be about 5% to about 5.01%, about 5% to about 5.02%, about 5% to about 5.03%, about 5% to about 5.04%, about 5% to about 5.05%, about 5% to about 5.06%, about 5% to about 5.07%, about 5% to about 5.08%, about 5% to about 5.09%, about 5% to about 5.1%, about 5.01% to about 5.02%, about 5.01% to about 5.03%, about 5.01% to about 5.04%, about 5.01% to about 5.05%, about 5.01% to about 5.06%, about 5.01% to about 5.07%, about 5.01% to about 5.08%, about 5.01% to about 5.09%, about 5.01% to about 5.1%, about 5.02% to about 5.03%, about 5.02% to about 5.04%, about 5.02% to about 5.05%, about 5.02% to about 5.06%, about 5.02% to about 5.07%, about 5.02% to about 5.08%, about 5.02% to about 5.09%, about 5.02% to about 5.1%, about 5.03% to about 5.04%, about 5.03% to about 5.05%, about 5.03% to about 5.06%, about 5.03% to about 5.07%, about 5.03% to about 5.08%, about 5.03% to about 5.09%, about 5.03% to about 5.1%, about 5.04% to about 5.05%, about 5.04% to about 5.06%, about 5.04% to about 5.07%, about 5.04% to about 5.08%, about 5.04% to about 5.09%, about 5.04% to about 5.1%, about 5.05% to about 5.06%, about 5.05% to about 5.07%, about 5.05% to about 5.08%, about 5.05% to about 5.09%, about 5.05% to about 5.1%, about 5.06% to about 5.07%, about 5.06% to about 5.08%, about 5.06% to about 5.09%, about 5.06% to about 5.1%, about 5.07% to about 5.08%, about 5.07% to about 5.09%, about 5.07% to about 5.1%,about 5.08% to about 5.09%, about 5.08% to about 5.1%, or about 5.09% to about 5.1%. The weight percentage may be about 5%, about 5.01%, about 5.02%, about 5.03%, about 5.04%, about 5.05%, about 5.06%, about 5.07%, about 5.08%, about 5.09%, or about 5.1%. The weight percentage may be at least about 5%, about 5.01%, about 5.02%, about 5.03%,about 5.04%, about 5.05%, about 5.06%, about 5.07%, about 5.08%, or about 5.09%. The weight percentage may be at most about 5.01%, about 5.02%, about 5.03%, about 5.04%, about 5.05%, about 5.06%, about 5.07%, about 5.08%, about 5.09%, or about 5.1%.

All other ranges recited in the above table include similar ranges and subranges and values within ranges. The present disclosure contemplates all similar ranges and subranges and values within ranges for each ingredient included in a formulation.

Four illustrative topical formulations are disclosed below:

Illustrative Topical Formulation 1

| Ingredient | Weight (%) |
|---|---|
| sodium bicarbonate | 33% |
| phosphatidylcholine | 4.03% |
| benzyl alcohol | 1.68% |
| isopropyl palmitate | 7.00% |
| stearic acid | 0.32% |
| cetyl alcohol | 2.00% |
| alcohol (ethanol) | 1.50% |
| Carthamus tinctorius (safflower) seed oil | 1.55% |
| oleic acid | 0.50% |
| almond oil | 3.00% |
| propylene glycol | 5.00% |
| water | 33.66% |
| glucose | 0.35% |
| poloxamer 407 | 5.40% |
| polyglyceryl-4 laurate | 1.00% |

Illustrative Topical Formulation 2

| Ingredient | Weight (%) |
|---|---|
| tris | 33% |
| phosphatidylcholine | 4.03% |
| benzyl alcohol | 1.68% |
| isopropyl palmitate | 7.00% |
| stearic acid | 0.32% |
| cetyl alcohol | 2.00% |
| alcohol (ethanol) | 1.50% |
| Carthamus tinctorius (safflower) seed oil | 1.55% |
| oleic acid | 0.50% |
| almond oil | 3.00% |
| propylene glycol | 5.00% |
| water | 33.66% |
| glucose | 0.35% |
| poloxamer 407 | 5.40% |
| polyglyceryl-4 laurate | 1.00% |

Illustrative Topical Formulation 3

| Ingredient | Weight (%) |
|---|---|
| sodium bicarbonate | 33% |
| phosphatidylcholine | 5.20% |
| benzyl alcohol | 0.95% |
| isopropyl palmitate | 9.05% |
| stearic acid | 0.42% |
| Carthamus tinctorius (safflower) seed oil | 1.99% |
| oleic acid | 0.46% |
| water | 41.42% |
| poloxamer 407 | 6.30% |
| polyglyceryl-4 laurate | 1.00% |

Illustrative Topical Formulation 4

| Ingredient | Weight (%) |
|---|---|
| calcium carbonate | 33% |
| phosphatidylcholine | 5.20% |
| benzyl alcohol | 0.95% |
| isopropyl palmitate | 9.05% |
| stearic acid | 0.42% |
| Carthamus tinctorius (safflower) seed oil | 1.99% |
| oleic acid | 0.46% |
| water | 41.42% |
| poloxamer 407 | 6.30% |
| polyglyceryl-4 laurate | 1.00% |

A topical formulation for transdermal delivery of a buffering agent comprises mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize one or more distinct permeation enhancers.

### Example 2 Methods for Treating a Treating or Preventing a Liver Disease

The present example relates to a method for treating a liver disease or reducing the risk of acquiring a liver disease. The method comprises administering to a subject in need thereof any herein-disclosed topical formulation for transdermal delivery of a buffering agent.

The topical formulation increases pH systemically in the subject and/or increases the subject's urine pH.

The liver disease may be a non-alcoholic fatty liver disease (NAFLD). The liver disease may be non-alcoholic steatohepatitis (NASH). The liver disease may be cirrhosis, hepatocellular carcinoma, hepatitis, alcoholic liver disease, hepatic ischemia/reperfusion injury from liver transplant or resection, Budd-Chiari syndrome, primary biliary cholangitis, primary sclerosing cholangitis, progressive familial intrahepatic cholestasis, and Wilson disease. The liver disease may be cirrhosis or the liver and/or hepatocellular carcinoma (HCC).

The subject may have insulin resistance and/or diabetes, e.g., Type 1 or Type 2 diabetes.

The subject may partake in a high-fat diet and/or partake in a high-calorie diet.

The subject may have a higher than recommended serum cholesterol and/or may have a higher than recommended serum triglyceride levels.

The subject may have an inflammatory disease and/or chronic inflammation. The subject may have increased expression of inflammatory cytokine, e.g., Interleukin 1 beta (IL-1β), Tumor Necrosis Factor alpha (TNF-α), Interleukin 6 (IL-6), and Transforming Growth Factor beta (TGF-β).

The subject may an abnormal gut microbiota.

A subject in need thereof is selected for having a liver disease or at risk for having a liver disease. In some cases, the subject in need thereof is not administered a topical formulation of the present disclosure for transdermal delivery of a buffering agent before being selected for having a liver disease or at risk for having a liver disease. In other words, the subject had not previously been administered a topical formulation of the present disclosure. Thus, the subject in need is specifically selected to be administered ministered a topical formulation of the present disclosure due a diagnosis of having a liver disease or at risk for having a liver disease.

In an embodiment, the period of administration of a topical formulation is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a topical formulation disclosed herein reduces signs/symptoms associated with a liver disease in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a topical formulation disclosed herein reduces signs/symptoms associated with a liver disease by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a topical formulation disclosed herein reduces signs/symptoms associated with a liver disease by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

### Example 3 Combination Therapies for Treating a Treating or Preventing a Liver Disease

Provided herein is a method for treating a liver disease or reducing the risk of acquiring a liver disease comprising administering to a subject in need thereof any herein-disclosed topical formulation for transdermal delivery of a buffering agent and administering an additional therapeutic for treating the liver disease or a related disorder.

The therapeutic for treating the liver disease or a related disorder may be administered before, contemporary with, or after the topical formulation.

The therapeutic may be administered orally, topically, enterally, parenterally, by intravenous injection or infusion, by intraperitoneal injection, by intramuscular injection, or by subcutaneous injection.

The therapeutic may be a liquid, a suspension, a gel, a geltab, a semisolid, a tablet, a sachet, a lozenge, a pill, or a capsule.

In some cases, the therapeutic is included in the topical formulation. And, transdermal delivery of the therapeutic via the topical formulation provides systemic administration of the therapeutic.

The therapeutic may be a drug relating to glycemic control (e.g., a sodium-glucose cotransporter-2 (SGLT2) inhibitor), a drug for lipid metabolism (e.g., a fibrate), and/or a NASH drug that has failed in a clinical trial due to lack of efficacy (e.g., elafibranor, cenicriviroc, remoglifozin, Obeticholic, Resmetirom, and Nalmefene).

In an embodiment, the period of administration of a topical formulation is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. The period of administration of the therapeutic (if distinct from the topical formulation) is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, the combination therapy reduces signs/symptoms associated with a liver disease in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, the combination therapy reduces signs/symptoms associated with a liver disease by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, the combination therapy reduces signs/symptoms associated with a liver disease by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

What is claimed is:

1. A method for treating a liver disease or reducing the risk of acquiring a liver disease, the method comprising administering to a subject in need thereof a topical formulation for transdermal delivery of a buffering agent,
wherein the topical formulation comprises a penetrant portion and a therapeutically effective amount of the buffering agent,
wherein the penetrant portion comprises: a phospholipid, a fatty acid ester formed from a low molecular weight alcohol, and a long-chain fatty acids, and, optionally, one or more of a viscosity-improving agents, a penetration enhancer, an oil, and an emulsifier, and
wherein the buffering agent comprises one or more of sodium bicarbonate (baking soda or sodium hydrogen carbonate), tris (tromethamine, trisaminomethane, 2-amino-2-hydroxymethyl-propane-1,3-diol, or tris(hydroxymethyl)aminomethane), calcium carbonate, tripotassium phosphate (potassium phosphate), sodium carbonate (disodium carbonate), sodium hydroxide (sodium oxidanide), potassium bicarbonate (potassium hydrogen carbonate or potassium acid carbonate), lysine, potassium carbonate, dipotassium phosphate (potassium phosphate dibasic or potassium hydrogen phosphate), disodium phosphate (sodium phosphate dibasic or disodium hydrogen phosphate), trisodium phosphate, meglumine ((2r,3r,4r,5s)-6-(methylamino)hexane-1,2,3,4,5-pentol or methylglucamine), arginine, triethanolamine (tea or 2,2′,2″-nitrilotriethanol), glycine, monosodium phosphate (sodium dihydrogen phosphate), monopotassium phosphate (potassium dihydrogen phosphate), monoethanolamine, diethanolamine (diolamine or 2-(2-hydroxyethylamino)ethanol), magnesium carbonate, and 2-imidazole-1-yl-3-ethoxycarbonylpropionic acid (IEPA).

2. The method of claim 1, wherein the phospholipid is selected from phosphatidylcholine, hydrogenated phosphatidylcholine, phosphatidylserine, phosphatidylethanolamine, phosphatidylinositol, inositol phosphatide, and sphingomyelin.

3. The method of claim 2, wherein the phospholipid is phosphatidylcholine.

4. The method of any one of claims 1 to 3, wherein the penetrant portion comprises two or more phospholipids.

5. The method of any one of claims 1 to 4, wherein the phospholipid is in an amount from about 3% to about 15% w/w of the topical formulation.

6. The method of any one of claims 1 to 5, wherein the low molecular weight alcohol is selected from isopropanol, methanol, ethanol, butanol, glycerol, cetyl alcohol.

7. The method of any one of claims 1 to 6, wherein the low molecular weight alcohol is isopropanol.

8. The method of any one of claims 1 to 7, wherein the fatty acid ester is selected from isopropyl palmitate, isopropyl myristate, isopropyl linoleate, isopropyl oleate, ethyl laurate, and ethyl myristate.

9. The method of claim 8, wherein the fatty acid ester is isopropyl palmitate.

10. The method of any one of claims 1 to 9, wherein the fatty acid ester is in an amount from about 5% to about 20% w/w of the topical formulation.

11. The method of any one of claims 1 to 10, wherein the fatty acid ester is in an amount from about 7% to about 9% w/w of the topical formulation.

12. The method of any one of claims 1 to 11, wherein the long-chain fatty acid is selected from a linoleic, oleic, stearic acid, linolenic, palmitic, arachidonic, palmitoleic, myristic, eicosenoic, benehic, euricic, and lignoceric acid.

13. The method of claim 12, wherein the long-chain fatty acid is linoleic acid or oleic acid.

14. The method of claim 12 or claim 13, wherein the long-chain fatty acid is stearic acid.

15. The method of any one of claims 1 to 14, wherein the long-chain fatty acid is in an amount from about 0.1% to about 2% w/w of the topical formulation.

16. The method of any one of claims 1 to 15, wherein the oil is safflower oil, macadamia oil, almond oil, another oil with high linoleic composition, or another oil with high oleic composition.

17. The method of any one of claims 1 to 16, wherein the oil is in an amount from about 1% to about 7% w/w of the topical formulation.

18. The method of any one of claims 1 to 17, wherein the oil is almond oil and in an amount from about 2.5% to about 3.5% w/w of the topical formulation.

19. The method of any one of claims 1 to 18, wherein the oil is safflower oil and in an amount from about 1.5% to about 2.5% w/w of the topical formulation.

20. The method of any one of claims 1 to 19, wherein the penetrant portion comprises a viscosity-improving agent.

21. The method of any one of claims 1 to 20, wherein the viscosity-improving agent is a poloxamer.

22. The method of claim 21, wherein the poloxamer is selected from poloxamer 407, poloxamer 188, poloxamer 184, and poloxamer 124.

23. The method of claim 22, wherein the poloxamer is in an amount from about 4% to about 7% w/w of the topical formulation.

24. The method of any one of claims 1 to 23, wherein the viscosity-improving agent is polyglyceryl-4 laurate.

25. The method of claim 24, wherein the polyglyceryl-4 laurate is in an amount from about 0.5% to about 2% w/w of the topical formulation.

26. The method of claim 24 or claim 25, wherein the polyglyceryl-4 laurate is in an amount of about 1% w/w of the topical formulation.

27. The method of any one of claims 1 to 26, wherein the penetrant portion comprises a penetration enhancer.

28. The method of any one of claims 1 to 27, wherein the penetration enhancer is an alcohol or a terpene.

29. The method of claim 28, wherein the penetration enhancer is an alcohol selected from benzyl alcohol, ethanol, propylene glycol, cetyl alcohol, and polyethylene glycol.

30. The method of claim 29, wherein the penetration enhancer comprises two or more alcohols.

31. The method of any one of claims 1 to 30, wherein the penetration enhancer further acts as a preservative.

32. The method of any one of claims 1 to 31, wherein the penetrant portion comprises two or more penetration enhancers.

33. The method of any one of claims 1 to 30, wherein the penetration enhancer is in an amount from about 1% to about 20% w/w of the topical formulation.

34. The method of any one of claims 1 to 33, wherein the penetration enhancer is in an amount from about 6% to about 15% w/w of the topical formulation.

35. The method of any one of claims 1 to 34, wherein the penetrant portion comprises at least one penetration enhancer and at least one viscosity-improving agent.

36. The method of any one of claims 1 to 35, wherein the penetrant portion comprises an emulsifier.

37. The method of any one of claims 1 to 36, wherein the emulsifier is selected from polyglyceryl-4-laurate, polyglyceryl-4-oleate, span 60, cetyl alcohol, and polyglyceryl-3-oleate.

38. The method of any one of claims 1 to 37, wherein the penetrant portion comprises two or more emulsifiers.

39. The method of any one of claims 1 to 38, wherein the emulsifier is in an amount from about 0.5 to about 10% w/w of the topical formulation.

40. The method of any one of claims 1 to 39, wherein the penetrant portion comprises at least one emulsifier and at least one viscosity-improving agent.

41. The method of any one of claims 1 to 40, wherein the penetrant portion comprises at least one emulsifier and at least one penetration enhancer.

42. The method of any one of claims 1 to 41, wherein the penetrant portion comprises at least one emulsifier, at least one viscosity-improving agent, and at least one penetration enhancer.

43. The method of any one of claims 1 to 41, wherein the topical formulation further comprises water.

44. The method of claim 43, wherein the water is in an amount from about 30% water to about 50% w/w of the topical formulation.

45. The method of claim 43 or 44, wherein the water is in an amount from about 33% water to about 42% w/w of the topical formulation.

46. The method of any one of claims 1 to 45, wherein the topical formulation further comprises glucose.

47. The method of claim 46, wherein the glucose is in an amount from about 0.25% to about 0.5% w/w of the topical formulation.

48. The method of claim 46 or 47, wherein the glucose is in an amount of about 0.55% w/w of the topical formulation.

49. The method of any one of claims 1 to 48, wherein the buffering agent is in an amount from about 25% to about 50% w/w of the topical formulation.

50. The method of claim 49, wherein the buffering agent is in an amount from about 30% to about 35% w/w of the topical formulation.

51. The method of claim 49 or 50, wherein the buffering agent is in an amount of about 33% w/w of the topical formulation.

52. The method of any one of claims 1 to 51, wherein the formulation is in the form of a cream, lotion, ointment, or the like.

53. The method of any one of claims 1 to 52, wherein the topical formulation increases pH systemically in the subject.

54. The method of any one of claims 1 to 53, wherein the topical formulation increases the subject's urine pH.

55. The method of any one of claims 1 to 54, wherein the liver disease is a non-alcoholic fatty liver disease (NAFLD).

56. The method of any one of claims 1 to 55, wherein the liver disease is a non-alcoholic steatohepatitis (NASH).

57. The method of any one of claims 1 to 56, wherein the liver disease is cirrhosis, hepatocellular carcinoma, hepatitis, alcoholic liver disease, hepatic ischemia/reperfusion injury from liver transplant or resection, Budd-Chiari syndrome, primary biliary cholangitis, primary sclerosing cholangitis, progressive familial intrahepatic cholestasis, and Wilson disease.

58. The method of any one of claims 1 to 57, wherein the subject has cirrhosis or the liver and/or hepatocellular carcinoma (HCC).

59. The method of any one of claims 1 to 58, wherein the subject has insulin resistance and/or diabetes, e.g., Type 1 or Type 2 diabetes.

60. The method of any one of claims 1 to 59, wherein the subject partakes in a high-fat diet and/or partakes in a high-calorie diet.

61. The method of any one of claims 1 to 60, wherein the subject has a higher than recommended serum cholesterol and/or has a higher than recommended serum triglyceride levels.

62. The method of any one of claims 1 to 61, wherein the subject has an inflammatory disease and/or chronic inflammation.

63. The method of any one of claims 1 to 62, wherein the subject has increased expression of inflammatory cytokines.

64. The method of claim 63, wherein the inflammatory cytokines are selected from Interleukin 1 beta (IL-1β), Tumor Necrosis Factor alpha (TNF-α), Interleukin 6 (IL-6), and Transforming Growth Factor beta (TGF-β).

65. The method of any one of claims 1 to 64, wherein the subject has an abnormal gut microbiota.

66. The method of any one of claims 1 to 65, wherein the subject is additionally administered a therapeutic for treating the liver disease or a related disorder.

67. The method of claim 66, wherein the therapeutic for treating the liver disease or a related disorder is administered before, contemporary with, or after the topical formulation.

68. The method of claim 66 or claim 67, wherein the therapeutic is administered orally, topically, enterally, parenterally, by intravenous injection or infusion, by intraperitoneal injection, by intramuscular injection, or by subcutaneous injection.

69. The method of any one of claims 66 to 68, wherein the therapeutic is a liquid, a suspension, a gel, a geltab, a semisolid, a tablet, a sachet, a lozenge, a pill, or a capsule.

70. The method of claim 66 or claim 67, wherein the therapeutic is included in the topical formulation.

71. The method of claim 70, wherein transdermal delivery of the therapeutic via the topical formulation provides systemic administration of the therapeutic.

72. The method of any one of claims 66 to 71, wherein the therapeutic is a drug relating to glycemic control (e.g., a sodium-glucose cotransporter-2 (SGLT2) inhibitor), a drug for lipid metabolism (e.g., a fibrate), and/or a NASH drug that has failed in a clinical trial due to lack of efficacy (e.g., elafibranor, cenicriviroc, remoglifozin, Obeticholic, Resmetirom, and Nalmefene).

73. The method of any one of claims 1 to 72, wherein the topical formulation comprises about 33% sodium bicarbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate.

74. The method of any one of claims 1 to 72, wherein the topical formulation comprises about 33% calcium carbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate; about 33% sodium bicarbonate, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate; or about 33% tris, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate.

75. The method of any one of claims 1 to 74, wherein the subject in need thereof is selected for having a liver disease or at risk for having a liver disease.

76. The method of claim 75, wherein the subject in need thereof is not administered the topical formulation for transdermal delivery of the buffering agent before being selected for having the liver disease.

77. A topical formulation for transdermal delivery of a buffering agent through the skin of a subject, the formulation comprising one or more buffering agents, one or more fatty acid esters, one or more alcohols, one or more long-chain fatty acids, one or more oils, one or more poloxamers, one or more phospholipids, polyglyceryl-4 laurate, and water.

78. The topical formulation of claim 77, wherein the one or more alcohols are present in an amount from about 1% to about 13% w/w of the topical formulation.

79. The topical formulation of claim 77 or claim 78, wherein the one or more alcohols are selected from benzyl alcohol, ethanol, cetyl alcohol, and propylene glycol.

80. The topical formulation of claim 79, wherein when present benzyl alcohol is in an amount from about 1% to about 2% w/w of the topical formulation; when present ethanol is in an amount from about 1% to about 2% w/w of the topical formulation; when present cetyl alcohol is in an amount from about 1.5% to about 2.5% w/w of the topical formulation; and when present propylene glycol is in an amount from about 4% to about 6% w/w of the topical formulation.

81. The topical formulation of any one of claims 77 to 80, wherein the one or more fatty acid esters are present in an amount from about 6% to about 10% w/w of the topical formulation.

82. The topical formulation of any one of claims 77 to 81, wherein the one or more fatty acid esters are present in an amount from about 7% to about 9% w/w of the topical formulation.

83. The topical formulation of any one of claims 77 to 82, wherein the one or more fatty acid esters is selected from isopropyl palmitate and isopropyl myristate.

84. The topical formulation of any one of claims 77 to 83, wherein the one or more long-chain fatty acids are present in an amount from about 0.3% to about 2% w/w of the topical formulation.

85. The topical formulation of any one of claims 77 to 84, wherein the one or more long the long-chain fatty acids are selected from a linoleic, oleic, stearic acid, linolenic, palmitic, arachidonic, palmitoleic, myristic, eicosenoic, benehic, euricic, and lignoceric acid.

86. The topical formulation of claim 85, wherein when present stearic acid is in an amount from about 0.3% to about 0.5% w/w of the topical formulation and when present oleic acid is in an amount from about 0.4% to about 0.6% w/w of the topical formulation.

87. The topical formulation of any one of claims 77 to 83, wherein the one or more oils are present in an amount from about 1% to about 7% w/w of the topical formulation.

88. The topical formulation of any one of claims 77 to 87, wherein the one or more oils are safflower oil, macadamia oil, almond oil, another oil with high linoleic composition, or another oil with high oleic composition.

89. The topical formulation of claim 88, wherein when present safflower oil is in an amount from about 1.5% to about 2% w/w of the topical formulation and when present almond oil is in an amount from about 2.5% to about 3.5% w/w of the topical formulation.

90. The topical formulation of any one of claims 77 to 89, wherein the one or more poloxamers are in an amount from about 4% to about 7% w/w of the topical formulation.

91. The topical formulation of any one of claims 77 to 90, wherein the one or more poloxamers are in an amount from about 5% to about 6.5% w/w of the topical formulation.

92. The topical formulation of any one of claims 77 to 91, wherein the one or more poloxamers are selected from poloxamer 407, poloxamer 188, poloxamer 184, and poloxamer 124.

93. The topical formulation of any one of claims 77 to 92, wherein the one or more phospholipids are in an amount from about 3.5% to about 4.5% w/w of the topical formulation.

94. The topical formulation of any one of claims 77 to 93, wherein the one or more phospholipids are selected from phosphatidylcholine, hydrogenated phosphatidylcholine, phosphatidylserine, phosphatidylethanolamine, phosphatidylinositol, inositol phosphatide, and sphingomyelin.

95. The topical formulation of any one of claims 77 to 94, wherein the polyglyceryl-4 laurate is in an amount from about 0.5% to about 1.5% w/w of the topical formulation.

96. The topical formulation of any one of claims 77 to 95, wherein the polyglyceryl-4 laurate is in an amount of about 1% w/w of the topical formulation.

97. The topical formulation of any one of claims 77 to 96, wherein the water is in an amount from about 30% to about 42% w/w of the topical formulation.

98. The topical formulation of any one of claims 77 to 97, wherein the one or more buffering agents are in an amount from about 25% to about 40% w/w of the topical formulation.

99. The topical formulation of any one of claims 77 to 98, wherein the one or more buffering agents are in an amount from about 30% to about 35% w/w of the topical formulation.

100. The topical formulation of any one of claims 77 to 99, wherein the one or more buffering agents are in an amount of about 33% w/w of the topical formulation.

101. The topical formulation of any one of claims 77 to 100, wherein the one or more buffering agents are selected from sodium bicarbonate (baking soda or sodium hydrogen carbonate), tris (tromethamine, trisaminomethane, 2-amino-2-hydroxymethyl-propane-1,3-diol, or tris(hydroxymethyl)aminomethane), calcium carbonate, tripotassium phosphate (potassium phosphate), sodium carbonate (disodium carbonate), sodium hydroxide (sodium oxidanide), potassium bicarbonate (potassium hydrogen carbonate or potassium acid carbonate), lysine, potassium carbonate, dipotassium phosphate (potassium phosphate dibasic or potassium hydrogen phosphate), disodium phosphate (sodium phosphate dibasic or disodium hydrogen phosphate), trisodium phosphate, meglumine ((2r,3r,4r,5s)-6-(methylamino)hexane-1,2,3,4,5-pentol or methylglucamine), arginine, triethanolamine (tea or 2,2′,2″-nitrilotriethanol), glycine, monosodium phosphate (sodium dihydrogen phosphate), monopotassium phosphate (potassium dihydrogen phosphate), monoethanolamine, diethanolamine (diolamine or 2-(2-hydroxyethylamino)ethanol), magnesium carbonate, and 2-imidazole-1-yl-3-ethoxycarbonylpropionic acid (IEPA).

102. The topical formulation of any one of claims 77 to 101, comprising about 33% sodium bicarbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate.

103. The topical formulation of any one of claims 77 to 101, comprising about 33% calcium carbonate, about 5.20% phosphatidylcholine, about 0.95% benzyl alcohol, about 9.05% isopropyl palmitate, about 0.42% stearic acid, about 1.99% Carthamus tinctorius (safflower) seed oil, about 0.46% oleic acid, about 41.42% water, about 6.30% poloxamer 407, and about 1.00% polyglyceryl-4 laurate.

104. The topical formulation of any one of claims 77 to 101, comprising about 33% sodium bicarbonate, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate.

105. The topical formulation of any one of claims 77 to 101, comprising about 33% tris, about 4.03% phosphatidylcholine, about 1.68% benzyl alcohol, about 7.00% isopropyl palmitate, about 0.32% stearic acid, about 2.00% cetyl alcohol, about 1.50% alcohol (ethanol), about 1.55% Carthamus tinctorius (safflower) seed oil, about 0.50% oleic acid, about 3.00% almond oil, about 5.00% propylene glycol, about 33.66% water, about 0.35% glucose, about 5.40% poloxamer 407, and about 1.00% polyglyceryl-4 laurate.

106. A method for treating a liver disease or reducing the risk of acquiring a liver disease, the method comprising a step of applying to the skin of a subject an effective amount of the topical formulation of any one of claims 77 to 105.

107. The method of claim 106, wherein the liver disease is a non-alcoholic fatty liver disease (NAFLD) with or without non-alcoholic steatohepatitis (NASH).

108. The method of claim 106 or claim 107, wherein the liver disease is cirrhosis, hepatocellular carcinoma, hepatitis, alcoholic liver disease, hepatic ischemia/reperfusion injury from liver transplant or resection, Budd-Chiari syndrome, primary biliary cholangitis, primary sclerosing cholangitis, progressive familial intrahepatic cholestasis, Wilson disease, and/or hepatocellular carcinoma (HCC).

109. The method of any one of claims 106 to 108, wherein the subject is additionally administered a therapeutic for treating the liver disease or a related disorder.

110. The method of claim 109, wherein the therapeutic for treating the liver disease or a related disorder is administered before, contemporary with, or after the topical formulation.

111. The method of claim 109 or claim 110, wherein the therapeutic is administered orally, topically, enterally, parenterally, by intravenous injection or infusion, by intraperitoneal injection, by intramuscular injection, or by subcutaneous injection.

112. The method of any one of claim 109 to claim 111, wherein the therapeutic is a liquid, a suspension, a gel, a geltab, a semisolid, a tablet, a sachet, a lozenge, a pill, or a capsule.

113. The method of claim 109, wherein the therapeutic is included in the topical formulation.

114. The method of claim 113, wherein transdermal delivery of the therapeutic via the topical formulation provides systemic administration of the therapeutic.

115. The method of any one of claims 109 to 114, wherein the therapeutic is a drug relating to glycemic control (e.g., a sodium-glucose cotransporter-2 (SGLT2) inhibitor), a drug for lipid metabolism (e.g., a fibrate), and/or a NASH drug that has failed in a clinical trial due to lack of efficacy (e.g., elafibranor, cenicriviroc, remoglifozin, Obeticholic, Resmetirom, and Nalmefene).

116. The method of any one of claims 106 to 115, wherein the subject in need thereof is selected for having a liver disease or at risk for having a liver disease.

117. The method of claim 116, wherein the subject in need thereof is not administered the topical formulation for transdermal delivery of a buffering agent before being selected for having a liver disease or at risk for having a liver disease.

118. Use of a topical formulation of any one of claims 77 to 105 in a method for treating a liver disease, reducing the risk of acquiring a liver disease, or reducing a symptom thereof.
